(19) United States
(12) Patent Application Publication
(10) Pub. No.: US 2024/0374626 A1
(43) Pub. Date: Nov. 14, 2024

(54) METHODS OF ENHANCING MILK PRODUCTION IN LACTATING MAMMALIAN SUBJECTS

(71) Applicant: The Regents of the University of California, Oakland, CA (US)

(72) Inventors: Lindsay Hinck, Santa Cruz, CA (US); Rut Molinuevo, Santa Cruz, CA (US); Julien Menendez, Santa Cruz, CA (US)

(21) Appl. No.: 18/646,322

(22) Filed: Apr. 25, 2024

Related U.S. Application Data
(60) Provisional application No. 63/462,326, filed on Apr. 27, 2023.

(51) Int. Cl. A61K 31/704; A61K 31/17; A61K 31/4709

(52) U.S. Cl. CPC A61K 31/704; A61K 31/17; A61K 31/4709

(57) ABSTRACT

Aspects of the present disclosure include methods for enhancing milk production in lactating mammalian subjects. In certain embodiments, the methods comprise administering a CDK1 inhibitor to mammary gland alveolar cells of a lactating mammalian subject in an amount effective to enhance milk production in the lactating mammalian subject. In some instances, the CDK1 inhibitor is a DNA damaging agent. The methods find use in enhancing milk production in a variety of lactating mammalian subjects. In certain embodiments, the subject is a human subject. In other embodiments, the subject is an ovine, caprine, or camelid subject.

### CROSS-REFERENCE TO RELATED APPLICATIONS

This application claims the benefit of U.S. Provisional Patent Application No. 63/462,326, filed Apr. 27, 2023, which application is incorporated herein by reference in its entirety.

### STATEMENT OF GOVERNMENT SUPPORT

This invention was made with Government support under contract R01 HD098722-01 awarded by the National Institutes of Health. The Government has certain rights in the invention.

### INCORPORATION BY REFERENCE OF SEQUENCE LISTING PROVIDED AS A SEQUENCE LISTING XML FILE APPLICATIONS

A Sequence Listing is provided herewith as a Sequence Listing XML, “UCSC-406_SEQLIST”, created on Jul. 19, 2024 and having a size of 16,023 bytes. The contents of the Sequence Listing XML are incorporated herein by reference in their entirety.

### INTRODUCTION

Endoreplication is the process by which a cell undergoes DNA replication in the absence of cell division, becoming polyploid. Developmentally programmed endoreplication occurs in several mammalian tissues during pregnancy and is usually linked to terminal differentiation. In the placenta, trophectoderm cells undergo endoreplication and differentiate into trophoblast giant cells, which penetrate the uterus and promote blastocyst implantation ¹⁻³. Subsequently, in the uterus, stromal cells of the endometrium endoreplicate and differentiate into decidual cells, which further facilitate blastocyst implantation and vascularization ⁴⁻⁶. Another example is pregnancy-induced liver growth occurring through hepatocyte hypertrophy that is generated by endoreplication ⁷. In the mammary gland (MG), alveolar cells undergo endoreplication at the onset of lactation ⁸⁻¹¹. While these phenomena have long been observed and considered adaptations necessary for tissue expansion during pregnancy, the molecular mechanisms driving these pregnancy-induced endoreplication events remain poorly understood.

The MG plays an essential role in the survival of mammalian species by producing milk required for the nourishment of offspring. During pregnancy, the MG undergoes a profound morphological change known as alveologenesis, in which epithelial luminal progenitors proliferate and subsequently differentiate into polyploid alveolar cells that secrete milk during lactation ¹². This polyploidization of the MG is conserved across many mammalian species, including mice and humans, and it is required for efficient milk production⁸⁻¹¹. Once breastfeeding is complete, in a process known as involution, massive cell death clears these milk-producing polyploid cells and tissue remodeling brings the epithelium back to a pre-pregnancy-like state.

The mechanisms by which endoreplication is achieved are diverse and vary between tissues. Endoreplication results in tetraploid cells (4C DNA content); however, cells can also undergo further endoreplication and become polyploid (>4C DNA content). This can be accomplished either by early mitotic arrest or cytokinetic failure ¹³. Endoreplication induced by early mitotic arrest occurs when a cell undergoes DNA replication without progressing through mitosis, becoming tetraploid and mononucleated. Endoreplication through early mitotic arrest requires inhibition of the Cyclin B/CDK1 complex that facilitates progression from the G2 phase to the M phase. In megakaryocytes, Cyclin B is downregulated during endoreplication leading to CDK1 inactivation ¹⁴. In other tissues, CDK1 is directly inactivated by CDK1 inhibitors. For example, in the placenta, the upregulation of the CDK1 inhibitor P57^{Kip2} in response to FGF4 deprivation induces trophoblast stem cells to differentiate into trophoblast giant cells and endoreplicate by preventing progression through mitosis ¹⁵. In the endometrium, upregulation of a different CDK1 inhibitor, P21^{Kip1}, has been suggested to inactivate CDK1 and induce G2/M arrest during the endoreplication of decidual cells ¹⁶. Alternatively, during endoreplication by cytokinetic failure, a cell progresses through mitosis unperturbed, but fails to divide, resulting in a tetraploid binucleated cell. These binucleated cells can arise in several ways, such as failure to specify a cleavage plane due to insufficient RhoA activation ¹⁷ or cleavage furrow ingression failure due to improper anchoring of the actomyosin ring ¹⁸. MG alveolar endoreplication has been suggested to require Aurora A kinase upregulation and cytokinesis failure ¹¹. Although the role of Aurora A during the G2/M transition and mitotic spindle assembly has been extensively studied, whether it is directly implicated in cytokinesis remains unclear ¹⁹. Therefore, the mechanisms regulating the transition from a proliferative mitotic cell cycle to an endocycle in the MG have yet to be elucidated.

The DNA damage response (DDR) plays a central role in the regulation of the cell cycle, to ensure genomic stability and safeguard inheritance. In the event of DNA damage, the DDR kinases ATM and ATR initiate a signaling cascade that activates cell cycle checkpoints, at either the G1/S or G2/M transitions, through inactivation of CDK/Cyclin complexes ²⁰. These checkpoints permit the DDR to perform any necessary repairs before giving rise to a daughter cell. DNA damage as a consequence of exogenous genotoxic insults has been shown to trigger endoreplication and terminal differentiation through G2/M checkpoint activation ²¹⁻²⁴.

### SUMMARY

Aspects of the present disclosure include methods for enhancing milk production in lactating mammalian subjects. In certain embodiments, the methods comprise administering a CDK1 inhibitor to mammary gland alveolar cells of a lactating mammalian subject in an amount effective to enhance milk production in the lactating mammalian subject. In some instances, the CDK1 inhibitor is a DNA damaging agent. The methods find use in enhancing milk production in a variety of lactating mammalian subjects. In certain embodiments, the subject is a human subject. In other embodiments, the subject is an ovine, caprine, or camelid subject.

### BRIEF DESCRIPTION OF THE FIGURES

FIG. 1: A heterogenous population of polyploid luminal cells emerges during lactation. a) Optical section from a 3D confocal image showing an alveolar unit from a MG at LD5 (left). Right image illustrates the heterogeneity of the MG by representing the DNA content of individual nuclei (2C in blue, >4C in yellow) calculated by in situ 3D DNA content analysis. E-cadherin detected in magenta. White broken line represents the border of the alveolus. Nuclear DNA by Hoechst. b) Optical section from a 3D confocal image at LD5 focused on a polyploid multinucleated alveolar cell. Arrows point to each of the 4 nuclei. E-cadherin shown in magenta. Nuclear DNA by Hoechst. c) FACS analysis of a representative MG single cell preparation used for the identification of the luminal CK8+ population. Red histogram represents the negative isotype control (IgG). Gate indicates the CK8+ population in relation to the negative isotype control. d) FACS analysis of the MG CK8+ cells for the identification of single cells, based on propidium iodide area (PI-A) versus width (PI-W). Black box shows the gating strategy for single cell identification. e) Representative FACS histograms for DNA content analysis of the CK8+ population in the nulliparous (left) and LD5 MGs (right). f) Quantification of FACS DNA content analysis showing the percentage of CK8+ cells with 2C, 4C or >4C DNA content, or in the S phase, from nulliparous, PD17.5, LD2 and LD5 MGs. g) Representative images and h) quantification of mononucleated and binucleated CK8+ cells from LD5 MGs, sorted based on DNA content (4C or >4C). Nuclear DNA by propidium iodide. Data are shown as the mean±SD; n=3 biological replicates. For (f) one-way ANOVA Tukey's test. p values: * <0.05, **<0.001, *** <0.0005, **** <0.0001.

FIG. 2: CDK1 inhibition increases endoreplication and milk production in HC11 and recapitulates the heterogeneity observed in the MG. a) Cartoon representing the differentiation process of HC11 cells. b) Quantification of FACS DNA content analysis showing the percentage of HC11 cells with 2C, 4C or >4C DNA content or in the S phase, 6 hours after treatment with DMSO or the Cdk1 inhibitor Ro-3306. c) Quantification of FACS DNA content analysis showing the percentage of differentiated HC11 cells (DIP3) with 2C, 4C or >4C DNA content or in the S phase, after treatment with DMSO or Ro-3306. d) Quantification of Csn2 expression in differentiated HC11 (DIP3) after DMSO or Ro-3306 treatment, as detected by RT-qPCR. e) Western Blot (left) and quantification (right) of CSN2 expression in differentiated HC11 (DIP3) after DMSO or Ro-3306 treatment. f) Quantification of percentage of mono-, bi- or multinucleated (more than 2 nuclei) differentiated HC11 (DIP3) treated with Ro-3306 or blebbistatin (blebbi) and sorted based on DNA content (4C or >4C). Data shown as the mean±SD except for (d) data shown as mean±S.E.M; n=3 biological replicates; two-tailed Student's t-test. p values: * <0.05, **<0.001, *** <0.0005, **** <0.0001.

FIG. 3: Physiological DNA damage occurs in the MG during pregnancy and regulates endoreplication and milk production in vivo. a) Detection of γH2AX (green) in optical-cleared MG sections at pregnancy day 10.5 (PD10.5). White broken lines indicate the single magnified alveolus that shows γH2AX in the luminal CK8⁺ cells (magenta). Nuclear DNA by propidium iodide. Images are representative of n=3 biological replicates. b) Quantification of γH2AX Integrated Density (IntDen) in the luminal CK8+ population from Nulliparous, PD6.5, PD10.5, PD15.5, PD18.5 and LD2 MGs. Data show values from 100 cells per time-point and are representative of n=3 biological replicates. Black horizontal bars represent the mean. Kruskal-Wallis with Dunn's multiple comparison test. c) Cartoon representing the contralateral intraductal injection (IDI) of MGs performed. Created with BioRender.com. d) Detection of γH2AX (green) at PD13.5, 24 hours after contralateral IDI with DMSO or doxorubicin (Doxo). CK8 shown in magenta. Nuclear DNA by propidium iodide. Images are representative of n=3 biological replicates. e) Quantification of γH2AX Integrated Density (IntDen) in the luminal CK8+ population after contralateral IDI with DMSO or Doxo. Data show values from 100 cells per treatment and are representative of n=3 biological replicates. Black horizontal bars represent the mean. Mann-Whitney test. f) Quantification of the percentage of CK8⁺ cells with >4C DNA content, detected by FACS analysis, after contralateral IDI with DMSO or Doxo in PD17.5 MGs. Data shown as the mean±SD; n=4 biological replicates; two-tailed paired Student's t-test. g) Quantification of Csn2 expression in PD17.5 MGs after contralateral IDI with DMSO or Doxo, as detected by RT-qPCR. Data shown as the mean±S.E.M; n=4 biological replicates; two-tailed paired Student's t-test. p values: * <0.05, **<0.001, **** <0.0001.

FIG. 4: The DNA damage response to replication stress is active in the MG during pregnancy. a) Detection of pATR (green) in optical-cleared MG sections at PD18.5. White broken lines indicate the single magnified alveolus that shows pATR in the luminal CK8⁺ (magenta) cells. Nuclear DNA by propidium iodide. Images are representative of n=3 biological replicates. b) Quantification of pATR Integrated Density (IntDen) in the luminal CK8+ (magenta) population from nulliparous (Nullip.), PD6.5, PD10.5, PD15.5, PD18.5 and LD2 MGs. Data show values from 100 cells per time-point and are representative of n=3 biological replicates. Black horizontal bars represent the mean. Kruskal-Wallis with Dunn's multiple comparison test. c and d) Quantification of γH2AX (c) or pATR (d) Integrated Density (IntDen) in undifferentiated wild-type (WT) or Cyclin E overexpressing (CCNE1) HC11. Data show values from 100 cells for WT or CCNE1 and are representative of n=3 biological replicates. Black horizontal bars represent the mean. Mann-Whitney test. e) Quantification of FACS DNA content analysis showing the percentage of differentiated (DIP3) WT or CCNE1 HC11 with 2C, 4C or >4C DNA content, or in the S phase. Data shown as the mean±SD; n=3 biological replicates; two-tailed unpaired Student's t-test. f) Western Blot (left) and quantification (right) of CSN2 expression in differentiated (DIP3) WT or CCNE1 HC11. Data shown as the mean±SD; n=3 biological replicates; two-tailed unpaired t-test. p values: * <0.05, **<0.001, *** <0.0005, **** <0.0001.

FIG. 5: Hydroxyurea induces replication stress and increases endoreplication and milk production in vivo. a) Cartoon representing the contralateral intraductal injection (IDI) of MGs performed. Created with BioRender.com. b) Detection of γH2AX (green, top) or pATR (green, bottom) in optical-cleared MG sections after contralateral IDI with PBS or hydroxyurea (Hu). CK8 shown in magenta. Nuclear DNA by propidium iodide. Images are representative of n=2 biological replicates. c and d) Quantification of γH2AX (c) or pATR (d) Integrated Density (IntDen) in the luminal CK8⁺ population after contralateral IDI with DMSO or Hu. Data show values from 100 cells per treatment and are representative of n=2 biological replicates. Black horizontal bars represent the mean. Mann-Whitney test. e) Quantification of the percentage of CK8⁺ cells with >4C DNA content, detected by FACS analysis, after contralateral IDI with PBS or Hu in LD2 MGs. Data shown as the mean±SD; n=4 biological replicates; two-tailed paired Student's t-test. f) Quantification of Csn2 expression in LD2 MGs after contralateral IDI with PBS or Hu, as detected by RT-qPCR. Data shown as the mean±S.E.M; n=4 biological replicates; two-tailed paired Student's t-test. g) Quantification of milk average Integrated Density (IntDen) per alveolus detected in LD2 MG alveoli after contralateral IDI with PBS or Hu. Data shown as the mean±S.E.M; n=4 for biological replicates; two-tailed Student's paired t-test. h and i) Detection of milk (white) in LD2 MGs after contralateral IDI with PBS or Hu. CK8 shown in magenta. Yellow squares indicate the magnified area shown in (i), illustrating the milk contained within the alveoli. Images are representative of n=4 independent experiments. p values: * <0.05, **** <0.0001.

FIG. 6: Nucleosides supplementation relieves replication stress and inhibits endoreplication and milk production. a) Cartoon representing the contralateral intraductal injection (IDI) of MGs performed. Created with BioRender.com. b) Detection of γH2AX (green, top) or pATR (green, bottom) in optical-cleared MG sections after contralateral IDI with PBS or nucleosides (Nucs). CK8 shown in magenta. Nuclear DNA by propidium iodide. Images are representative of n=3 biological replicates. c and d) Quantification of γH2AX (c) or pATR (d) Integrated Density (IntDen) in the luminal CK8+ population after contralateral IDI with DMSO or Nucs. Data show values from 100 cells per treatment and are representative of n=3 biological replicates. Black horizontal bars represent the mean. Mann-Whitney test. e) Quantification of the percentage of CK8+ cells with 2C and 4C DNA content, detected by FACS analysis, after contralateral IDI with PBS or Nucs in LD2 MGs. Data shown as the mean±SD; n=4 biological replicates; two-tailed paired Student's t-test. f) Quantification of the percentage of CK8+ cells with >4C DNA content, detected by FACS analysis, after contralateral IDI with PBS or Nucs in LD5 MGs. Data shown as the mean±SD; n=3 biological replicates; two-tailed paired Student's t-test. g) Quantification of Csn2 expression in LD2 or LD5 MGs after contralateral IDI with PBS or Nucs, as detected by RT-qPCR. Data shown as the mean±S.E.M; n=4 for LD2 and n=3 for LD5 biological replicates; two-tailed Student's paired t-test. h) Quantification of milk average Integrated Density (IntDen) per alveolus detected in LD5 MG alveoli after contralateral IDI with PBS or Nucs. Data shown as the mean±S.E.M; n=3 for biological replicates; two-tailed Student's paired t-test. i and j) Detection of milk (white) in LD5 MGs after contralateral IDI with PBS or Nucs. CK8 shown in magenta. Yellow squares indicate the magnified area shown in (j), illustrating the milk contained within the alveoli. Images are representative of n=3 independent experiments. p values: * <0.05, **<0.001, **** <0.0001.

FIG. 7: Pharmacological inhibition of WEE1 decreases endoreplication and milk production during lactation. a) Quantification of Wee1 expression in whole MG tissue from nulliparous, PD6.5, PD10.5, PD15.5, PD18.5 and LD2 MGs, as detected by RT-qPCR. Data shown as the mean±S.E.M; n=4 biological replicates. one-way ANOVA Tukey's test. b) Quantification of Wee1 expression in sorted luminal, basal and stromal populations from PD18.5 and LD2 MGs, as detected by RT-qPCR. Populations were FACS-sorted based on expression of CD24 and CD29. Data shown as the mean±S.E.M; n=3 biological replicates. two-tailed paired Student's t-test. c) Quantification of Wee1 expression in whole MG tissue after contralateral IDI with DMSO/Doxo, PBS/Hu or PBS/Nucs. Data shown as the mean±S.E.M; n=4 biological replicates. two-tailed paired Student's t-test. d) Cartoon representing the contralateral intraductal injection (IDI) of MGs performed. Created with BioRender.com. e) Quantification of the percentage of CK8⁺ cells with 2C and 4C DNA content, detected by FACS analysis, after contralateral IDI with DMSO or Mk-1775 in LD2 MGs. Data shown as the mean±SD; n=4 biological replicates; two-tailed paired Student's t-test. f) Quantification of the percentage of CK8⁺ cells with >4C DNA content, detected by FACS analysis, after contralateral IDI with DMSO or MK-1775 in LD5 MGs. Data shown as the mean±SD; n=3 biological replicates; two-tailed paired Student's t-test. g and h) Detection of milk (white) in LD5 MGs after contralateral IDI with DMSO or Mk-1775. CK8 shown in magenta. Yellow squares indicate the magnified area shown in (h), illustrating the milk contained within the alveoli. Images are representative of n=4 independent experiments. i) Quantification of milk average Integrated Density (IntDen) per alveolus, detected by IHC, after contralateral IDI with DMSO or Mk-1775. Data shown as the mean±S.E.M; n=4 biological replicates; two-tailed paired Student's t-test. j) Quantification of Csn2 expression in LD2 or LD5 MGs after contralateral IDI with DMSO or Mk-1775, as detected by RT-qPCR. Data shown as the mean±S.E.M; n=4 biological replicates; two-tailed paired Student's t-test. p values: * <0.05, **<0.001, **** <0.0001.

FIG. 8: WEE1 regulates alveolar endoreplication and milk production during lactation. a) Quantification of FACS DNA content analysis showing the percentage of CK8⁺ cells with 2C, 4C or >4C DNA content in LD5 MGs from Ck8-CreER/mTmG/Wee1^{+/+} (Wee1^{+/+}) or Ck8-CreER/mTmG/Wee1^{fl/+} (Wee1^{fl/+}) mice. Data shown as the mean±SD; n=3 biological replicates; two-tailed unpaired Student's t-test. b) Quantification of Csn2 expression in LD5 MGs from Wee1^{+/+} or Wee1^{t/+} mice, as detected by RT-qPCR. Data shown as the mean±S.E.M; n=3 biological replicates; two-tailed unpaired Student's t-test. c) Quantification of milk average Integrated Density (IntDen) per alveolus detected by IHC in Wee1^{+/+} or Wee1^{fl/+} mice at LD5. Data shown as the mean±S.E.M; n=3 for biological replicates; two-tailed paired Student's t-test. d and e) Detection of milk (white) in LD5 MGs from Wee1^{+/+} or Wee1^{fl/+} mice. CK8 shown in magenta. Yellow squares indicate the magnified area shown in (e), illustrating the milk contained within the alveoli. Images are representative of n=3 independent experiments. f) Cartoon representing the regulation of alveolar endoreplication by the ATR-DNA damage response pathway. Proliferation of alveolar cells results in accumulation of DNA damage during pregnancy and activation of the ATR pathway. As a result, ATR mediates the transition from a mitotic cell cycle into an endocycle through the activation of the CDK1 inhibitor WEE1, which activates the G2/M checkpoint and regulates alveolar endoreplication during lactation. p values: * <0.05, **<0.001.

### DETAILED DESCRIPTION

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods of Enhancing Milk Production

Aspects of the present disclosure include methods for enhancing milk production in lactating mammalian subjects. In certain embodiments, the methods comprise administering a CDK1 inhibitor to mammary gland alveolar cells of a lactating mammalian subject in an amount effective to enhance milk production in the lactating mammalian subject. By “enhance” milk production is meant milk production in the subject is enhanced (increased) as compared to milk production in the subject in the absence of administration of the CDK1 inhibitor.

DNA damage as a consequence of exogenous genotoxic insults has been shown to trigger endoreplication and terminal differentiation through G2/M checkpoint activation. The methods of the present disclosure are based in part on the surprising identification of an unconventional trigger, physiological DNA damage, which as demonstrated herein accumulates during the extensive cell proliferation of mid-pregnancy and drives these events at the onset of lactation. This mechanism involves the activation of the ATR-mediated DDR to replication stress, and the subsequent activation of the G2/M checkpoint. WEE1 governs this process, revealing a novel role for this CDK1 inhibitor in the regulation of mammalian endoreplication. Details regarding the methods of the present disclosure are provided below.

Any agent that finds use in inhibiting CDK1 in a mammalian subject may be employed. In certain embodiments, the agent is a small molecule compound. By “small molecule” compound is meant a compound having a molecular weight of 1000 atomic mass units (amu) or less. In some embodiments, the small molecule is 900 amu or less, 750 amu or less, 500 amu or less, 400 amu or less, 300 amu or less, or 200 amu or less. In some instances, the small molecule is not made of repeating molecular units such as are present in a polymer. Small molecule inhibitors of CDK1 are known. Non-limiting examples of such inhibitors which may be employed when practicing the methods of the present disclosure include RO-3306, BA-j, and the like.

In certain embodiments, the CDK1 inhibitor is an agent that increases expression of Wee1-like protein kinase (WEE1) as compared to WEE1 expression in the absence of the agent. Examples of such agents include an expression construct comprising a nucleic acid encoding WEE1 operably linked to a suitable promoter, for delivery and expression of WEE1 in mammary gland alveolar cells of the subject. In some instances, the agent upregulates expression of WEE1, such as a transcriptional activator of the endogenous gene encoding WEE1.

According to some embodiments, the CDK1 inhibitor is a DNA damaging agent. Non-limiting examples of DNA damaging agents that may be employed when practicing the methods of the present disclosure include those described, e.g., in Cheung-Ong et al. (2013) Chem Biol. 20(5):648-59; Brown et al. (2018) Br J Cancer 118(3):312-324; and Daley et al. (2022) Front Oncol. 12:1048705; and Costa de Almeida et al. (2021) Cancer Genet. 252-253:6-24; the disclosures of which are incorporated herein by reference in their entireties for all purposes.

In some instances, the DNA damaging is a DNA intercalating agent. According to some embodiments, the DNA intercalating agent is an anthracycline. A non-limiting example of an anthracycline that may be employed is doxorubicin.

In certain embodiments, the DNA damaging agent is an inhibitor of ribonucleoside diphosphate reductase. By way of example, the DNA damaging agent may be hydroxyurea.

According to some embodiments, the DNA damaging agent is a reactive oxygen species, UV light, ionizing radiation, and/or the like.

In some instances, the CDK1 inhibitor is an agent that reduces expression of CDK1 as compared to CDK1 expression in the absence of the agent. In certain embodiments, such an agent is a nucleic acid-based inhibitor of CDK1 expression. By “nucleic acid-based inhibitor” is meant a polymer of two or more linked nucleotides, where the polymer may include naturally occurring nucleotides, non-naturally occurring nucleotides (e.g., nucleotide analogs such as LNA, FANA, 2′-O-Me RNA, 2′-fluoro RNA, and/or the like), or a combination thereof.

In some embodiments, a nucleic acid-based CDK1 inhibitor includes a region complementary to a portion of a messenger RNA (mRNA) that encodes CDK1. The term “complementary” as used herein refers to a nucleotide sequence that base-pairs by non-covalent bonds to all or a region of a target nucleic acid. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a primer may be perfectly (i.e., 100%) complementary to the target nucleic acid, or the primer and the target nucleic acid may share some degree of complementarity which is less than perfect (e.g., 70%, 75%, 85%, 90%, 95%, 99%). The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=#of identical positions/total #of positions ×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

Non-limiting examples of nucleic acid-based inhibitors that may be employed when practicing the methods of the present disclosure include short interfering RNAs (siRNA), microRNAs (miRNA), morpholinos, and/or the like. Based on the available sequence information for CDK1 (Gene ID: 983), and the corresponding transcript, nucleic acid-based inhibitors such as siRNAs, miRNAs, morpholinos, etc. may be designed using available tools, e.g., siRNA Wizard from Invivogen, siDESIGN Center from Dharmacon, BLOCK-iT™ RNAi Designer from Invitrogen, miR-Synth available at microrna.osumc.edu/mir-synth, WMD3—Web MicroRNA Designer, a morpholino design tool provided by Gene Tools, etc. Approaches for designing and delivering siRNAs, miRNAs, morpholinos, etc. for targeting a particular mRNA are known and described, e.g., in Chakraborty et al. (2017) Mol Ther Nucleic Acids 8:132-143; Ahmadzada et al. (2018) Biophys Rev. 10(1):69-86; Zheng et al. (2018) Trends Biotechnol. 36(5):562-575; Mohanty et al. (2015) Curr Pharm Des. 21(31):4606-13; Gomes et al. (2015) Ageing Res Rev. 21:43-54; Gustincich et al. (2017) Prog Neurobiol. 155:194-211; Monsoori et al. (2014) Adv Pharm Bull. 4(4):313-321; and Xin et al. (2017) Mol Cancer 16:134; the disclosures of which are incorporated herein by reference in their entireties for all purposes.

The CDK1 inhibitor may be administered via any route of administration suitable for delivery to the mammary gland alveolar cells of the subject. In certain embodiments, the CDK1 inhibitor is administered parenterally, e.g., by intravenous, intra-arterial, intra-mammary gland, or subcutaneous administration. In some instances, when the CDK1 inhibitor is administered via a parenteral route of administration, the CDK1 inhibitor is intraductally administered to the subject. For example, the administration may be by intraductal injection (IDI). Approaches for IDI administration are known and described, e.g., in Wang et al. (2022) PNAS 119 (24) e2200200119; Murata et al. (2006) Cancer Res. 66:638-645; Stearns et al. (2011) Sci. Transl. Med. 3, 106ra108; and Joseph et al. (2022) Mol. Pharm. 17, 441-452; the disclosures of which are incorporated herein by reference in their entireties for all purposes.

In some instances, the methods find use in treating a lactation insufficiency in the subject. By “treat” or “treatment” is meant at least an amelioration of one or more symptoms associated with the condition of the subject (e.g., lactation insuffic, ie.iency), where amelioration is used in a broad sense to refer to at least a reduction in the magnitude of a parameter, e.g., symptom, associated with the condition being treated. As such, treatment also includes situations where the condition, or at least one or more symptoms associated therewith, are reduced or completely inhibited, e.g., prevented from happening, or stopped, e.g., terminated, such that the subject no longer suffers from the condition, or at least the symptoms that characterize the condition.

The CDK1 inhibitor may be administered (e.g., in a pharmaceutical composition) in an effective amount. By “effective amount” is meant a dosage sufficient to produce a desired result, e.g., an amount sufficient to effect beneficial or desired therapeutic (including preventative) results, such as a reduction in a symptom of a lactation deficiency, as compared to a control. An effective amount can be administered in one or more administrations. The CDK1 inhibitor is generally administered to mammary gland alveolar cells of the subject in an amount effective to enhance milk production in the lactating mammalian subject, i.e., as compared to milk production in the lactating mammalian subject in the absence of administration of the CDK1 inhibitor.

For purposes of completeness, the present disclosure is further defined in the following numbered clauses.

1. A method for enhancing milk production in a lactating mammalian subject, the method comprising:
administering a CDK1 inhibitor to mammary gland alveolar cells of the subject in an amount effective to enhance milk production in the lactating mammalian subject.
   2. The method according to clause 1, wherein the CDK1 inhibitor is an agent that increases expression of Wee1-like protein kinase (WEE1) as compared to WEE1 expression in the absence of the agent.
   3. The method according to clause 1 or clause 2, wherein the CDK1 inhibitor is a DNA damaging agent.
   4. The method according to clause 3, wherein the DNA damaging agent is a DNA intercalating agent.
   5. The method according to clause 4, wherein the DNA intercalating agent is an anthracycline.
   6. The method according to clause 5, wherein the anthracycline is doxorubicin.
   7. The method according to clause 3, wherein the DNA damaging agent is an inhibitor of ribonucleoside diphosphate reductase.
   8. The method according to clause 7, wherein the DNA damaging agent is hydroxyurea.
   9. The method according to clause 1, wherein the CDK1 inhibitor is an agent that reduces expression of CDK1.
   10. The method according to clause 9, wherein the CDK1 inhibitor is a nucleic acid-based inhibitor comprising a region complementary to a portion of a messenger RNA (mRNA) that encodes CDK1.
   11. The method according to clause 10, wherein the nucleic acid-based inhibitor is a short interfering RNA (siRNA), a microRNA (miRNA), or a morpholino.
   12. The method according to any one of clauses 1 to 11, wherein the CDK1 inhibitor is parenterally administered to the lactating mammalian subject.
   13. The method according to clause 12, wherein the CDK1 inhibitor is intraductally administered to the lactating mammalian subject.
   14. The method according to any one of clauses 1 to 13, wherein the lactating mammalian subject is a lactating human subject.
   15. The method according to any one of clauses 1 to 13, wherein the lactating mammalian subject is a lactating bovine subject.
   16. The method according to any one of clauses 1 to 13, wherein the lactating mammalian subject is a lactating ovine, caprine, or camelid subject.

The following examples are offered by way of illustration and not by way of limitation.

### EXPERIMENTAL

### Introduction

Lactation insufficiency affects many women worldwide. During lactation, a large portion of mammary gland alveolar cells become polyploid, but how these cells balance the hyperproliferation occurring during normal alveologenesis with terminal differentiation is unknown. As demonstrated herein, DNA damage accumulates due to replication stress during pregnancy, activating the ATR-DNA damage response pathway. Modulation of DNA damage levels in vivo by intraductal injections of nucleosides or DNA damaging agents demonstrates that the degree of DNA damage accumulated during pregnancy governs endoreplication and milk production. Identified is a mechanism involving early mitotic arrest through CDK1 inactivation, resulting in a heterogeneous alveolar population with regards to ploidy and nuclei number. The inactivation of CDK1 is mediated by the DNA damage response kinase WEE1 with heterozygous loss of Wee1 resulting in decreased endoreplication and milk production. These results indicate that the DNA damage response to replication stress couples proliferation and endoreplication during mammary gland alveologenesis. The present examples shed light on mechanisms governing lactogenesis and identifies non-hormonal means for increasing milk production.

### Example 1—Endoreplication Results in a Heterogeneous Alveolar Population During Lactation

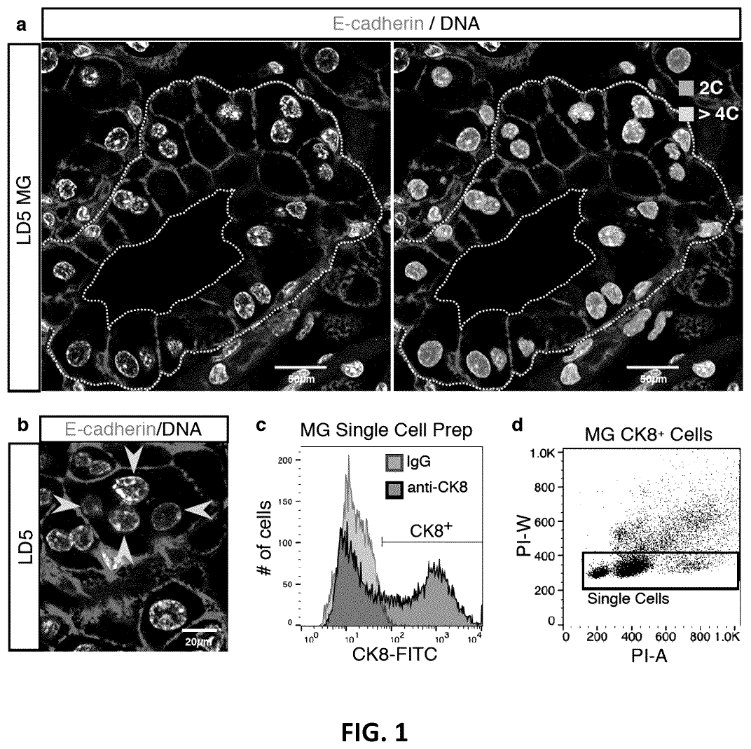

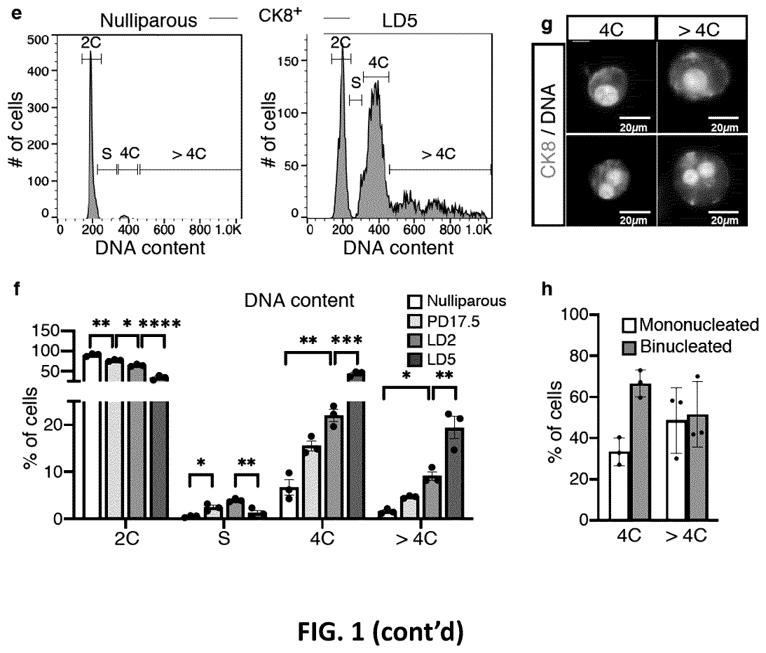

Mammary alveolar cells have previously been shown to undergo endoreplication during lactation, a process essential for efficient milk production ⁸⁻¹¹. How these cells become committed to endoreplication and the outcome of this endoreplication (in terms of DNA content and number of nuclei) remains unclear. It has previously been shown that a significant percentage of alveolar cells become tetraploid (4C) and binucleated during lactation ^{11,25}. In the present study, through IHC staining and in situ 3D DNA content analysis of tissue sections from lactation day (LD) 5 MGs, polyploid (>4C) mononucleated alveolar cells and binucleated cells containing polyploid nuclei were further detected (FIG. 1a). Also detected was the rare occurrence of multinucleated cells (FIG. 1b, arrows). To better understand the heterogeneity of the alveolar population, FACS DNA content analysis of the Cytokeratin-8 positive (CK8⁺) luminal cell population from MGs of nulliparous, pregnancy day (PD) 17.5, LD2 and LD5 mice was performed (FIG. 1c-f and Suppl. FIG. 1a). Consistent with previous reports ¹¹, a substantial increase in the proportion of tetraploid cells during lactation was observed (FIG. 1e, f). In addition, identified here is a novel polyploid subpopulation that arises at the end of pregnancy and expands during lactation (FIG. 1e, f and Suppl. FIG. 1a). Through visualization of the FACS-purified CK8⁺ population from LD5 MGs, ˜35% of tetraploid cells were found to be mononucleate, with the remaining ˜65% binucleate, whereas polyploid cells were observed to be both mononucleate and binucleated in a ˜50/50 ratio (FIG. 1g, h). Together, these results demonstrate mammary alveolar cells are heterogeneous during lactation with respect to DNA content and nuclei number.

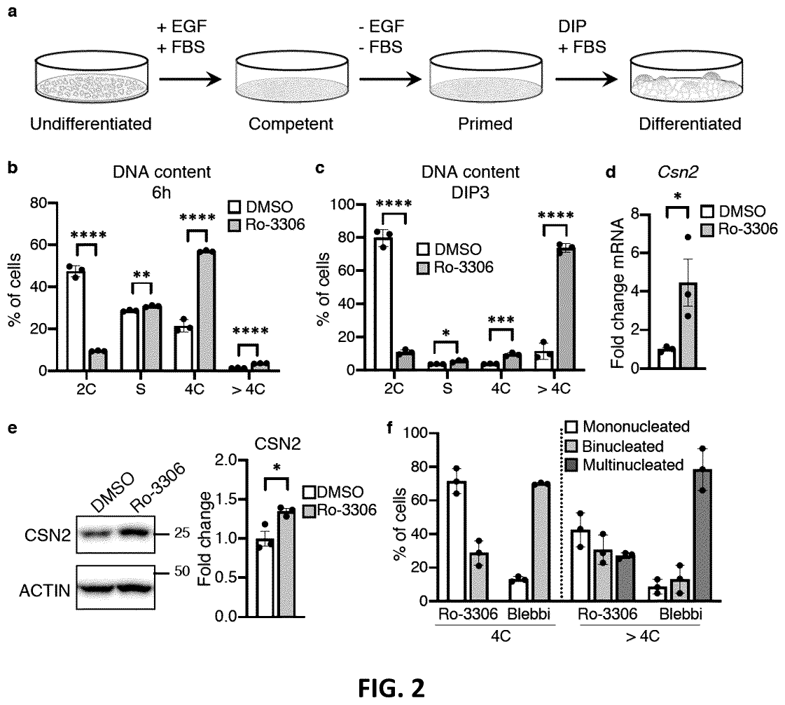

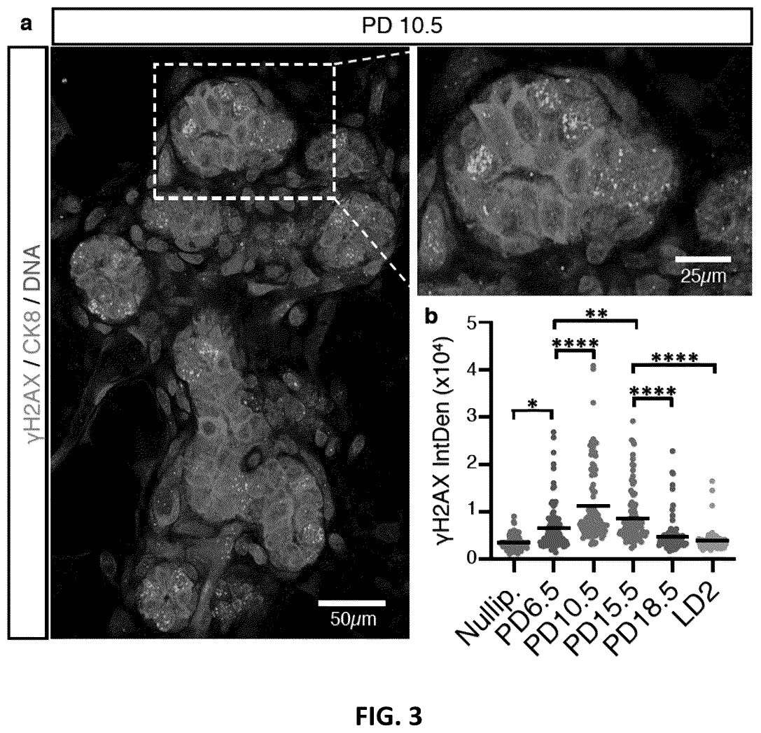

To further investigate the role of endoreplication during alveologenesis, the HC11 murine mammary cell line was employed as an in vitro lactation model. This cell line resembles the MG in that it undergoes differentiation into milk-producing secretory cells when cultured in the presence of the lactogenic hormones dexamethasone, insulin, and prolactin (DIP, FIG. 2a). As previously published ²⁶, ˜80% of differentiated HC11 cells were found to undergo G0/G1 arrest, presenting 2C DNA content. In addition, identified here was a population of polyploid HC11 cells that increases during differentiation. This observation suggests HC11 cells undergo endoreplication during differentiation, in accordance with the role of endoreplication in milk production. To further investigate this, undifferentiated HC11 cells at 80% confluence were treated with blebbistatin (Blebbi, 30 uM), a myosin II inhibitor that prevents cytokinesis and induces endoreplication through cytokinesis failure ²⁷. FACS DNA content analysis 6 hours post-treatment shows blebbistatin efficiently induces mitotic arrest in HC11 cells, increasing the proportion of tetraploid cells. By differentiation day 3 (DIP3), cells escape the mitotic arrest imposed by blebbistatin and undergo further endoreplication, becoming polyploid (Suppl. FIG. 2b). Increased polyploidy is accompanied by an increase in the formation of milk-containing domes, and increased expression of the milk proteins β-Casein (Csn2) and Periplin-2 (PLIN2), detected by RT-qPCR and ICC respectively (Suppl. FIG. 2c-f). Together, these data demonstrate HC11 cells endoreplicate when cell division is blocked, resulting in increased milk production. They are, therefore, a suitable in vitro model to investigate endoreplication during alveolar differentiation.

### Example 2—CDK1 Inhibition Drives Endoreplication of Alveolar Cells Through an Early Mitotic Arrest

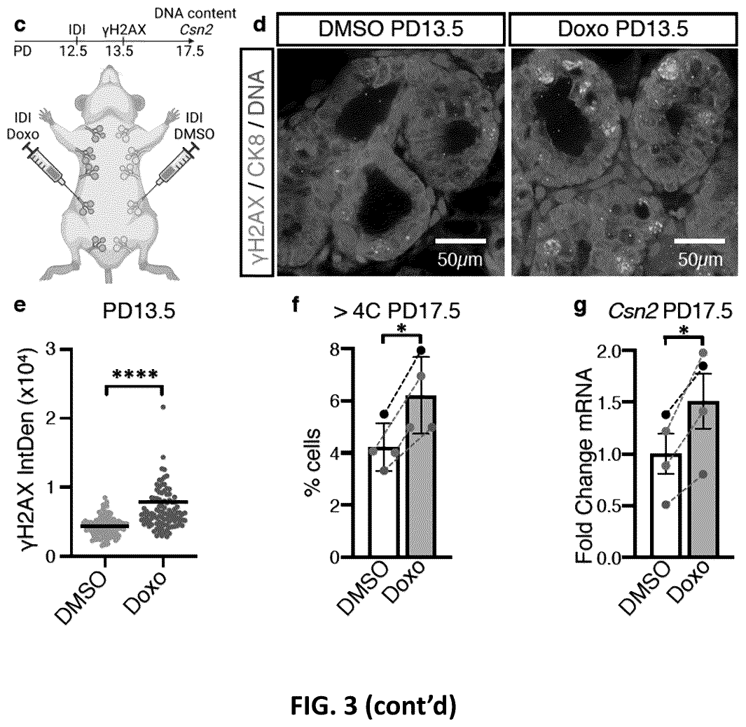

Through visualization of FACS-purified subpopulations, it was determined that a large proportion of polyploid HC11 cells were mononucleate at DIP3. While cytokinetic failure has been suggested to generate binucleated alveolar cells in vivo ¹¹, the presence of mononucleate polyploid cells both in vitro and in vivo indicates an early mitotic arrest at the G2/M transition is also a contributing factor. Endoreplication through early mitotic arrest requires the inactivation of the mitotic regulator CDK1²⁸. In addition, there must be a transition in the activity of CDK/CYCLIN complexes. CYCLIN B must be downregulated to facilitate CDK1 inactivation and G2/M arrest, while the activity of the CDK2/CYCLIN E complex must persist to allow for DNA replication ²⁹. Accordingly, during lactogenic differentiation of HC11 cells, CYCLIN B expression was found to be lost while CYCLIN E expression is maintained. To investigate if CDK1 inactivation is sufficient to induce endoreplication during lactogenic differentiation, undifferentiated HC11 at 80% confluence were treated with the CDK1 inhibitor Ro-3306 (5 μM). FACS DNA content analysis 6 hours after treatment with Ro-3306 shows CDK1 inhibition efficiently induces mitotic arrest, as detected by the accumulation of tetraploid cells (FIG. 2b and Suppl. FIG. 3c). By DIP3, cells escape the mitotic arrest imposed by Ro-3306 and undergo further endoreplication, becoming polyploid (FIG. 2c and Suppl. FIG. 3d). Accordingly, CSN2 expression increases, as detected by RT-qPCR and WB (FIG. 2d, e). These results show CDK1 inhibition and early mitotic arrest are sufficient to drive endoreplication during lactogenic differentiation in HC11 cells, resulting in increased milk production. Through visualization of FACS-purified subpopulations, it was observed that CDK1 inhibition generates mono-, bi- and multinucleated polyploid cells (FIG. 2f). In contrast, treatment with blebbistatin predominantly generates bi- and multinucleated cells (FIG. 2f). This shows that the heterogeneity of the MG observed in vivo is recapitulated by CDK1 inhibition, but not by the cytokinesis failure induced by blebbistatin. Altogether, these results suggest early mitotic arrest imposed by CDK1 inhibition is involved in alveolar endoreplication.

### Example 3—DNA Damage During Alveologenesis Increases Mammary Alveolar Endoreplication

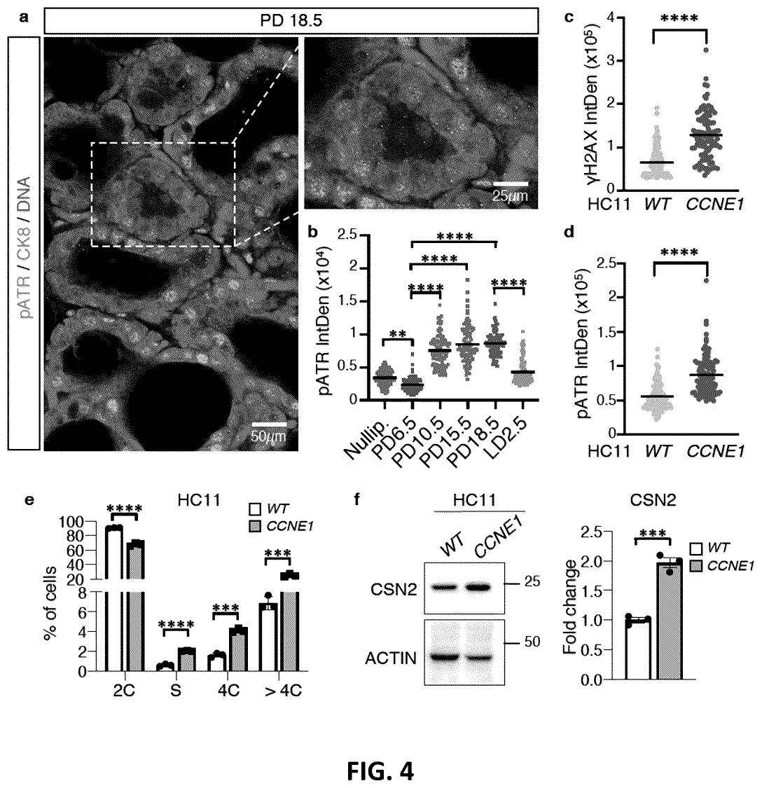

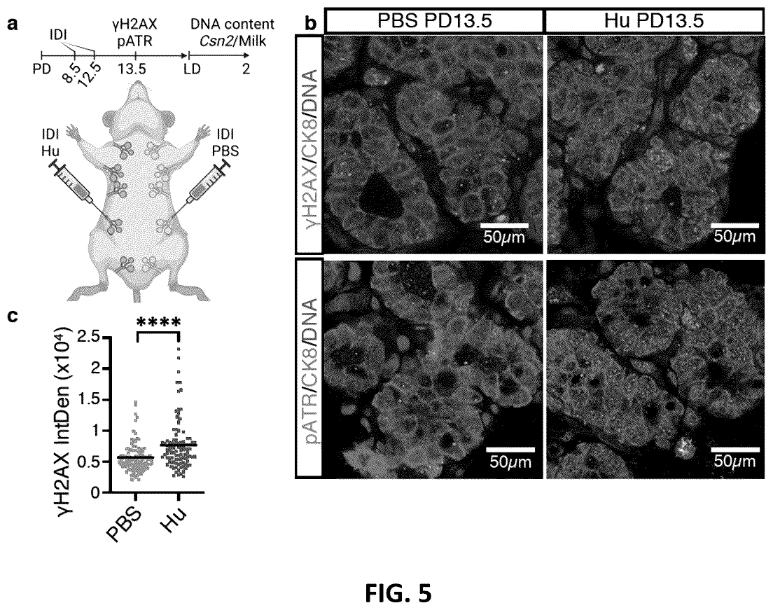

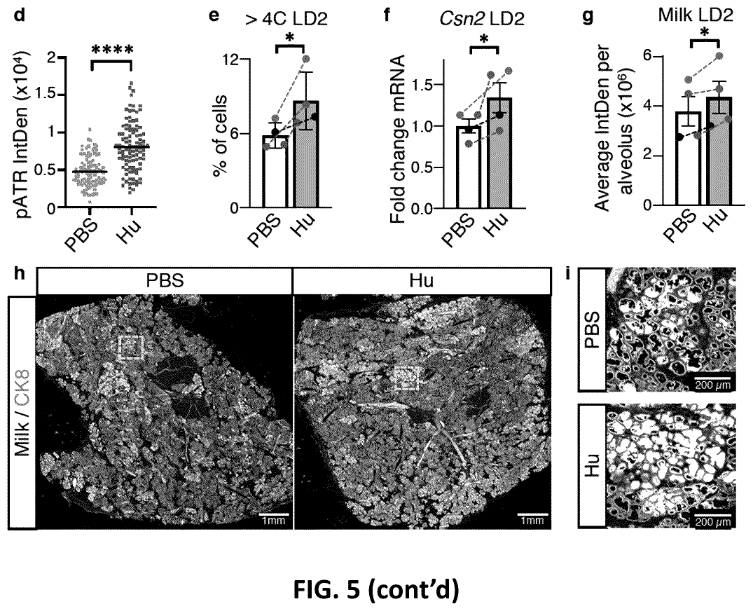

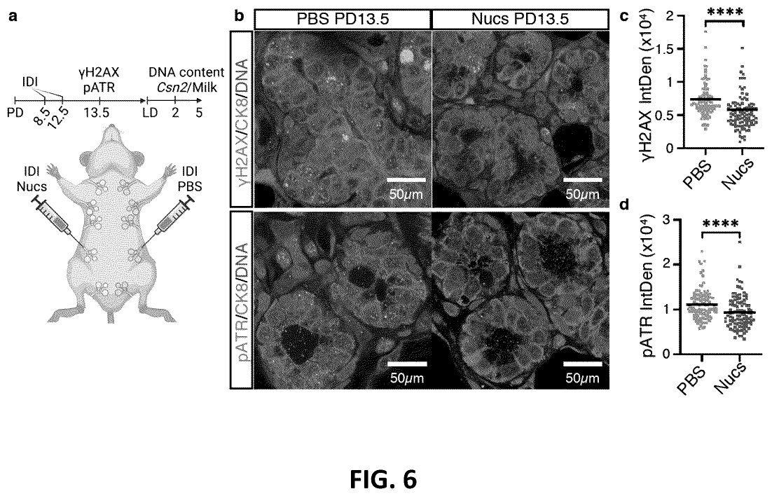

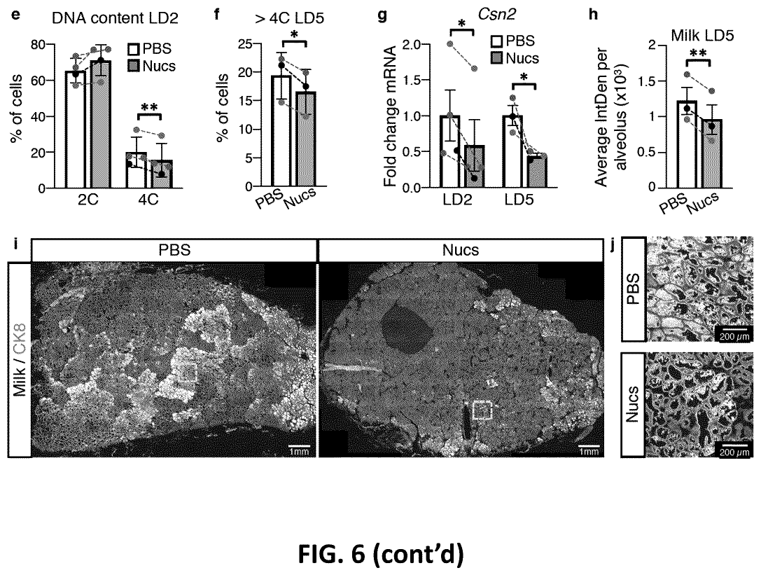

Developmentally programmed endoreplication occurs in different mammalian tissues not only during pregnancy ¹⁻¹¹, but also during organogenesis and tissue regeneration in response to injury ^{21,22,30-34}. In addition, DNA damage induced by genotoxic stress has been shown to induce endoreplication and terminal differentiation through the activation of the G2/M cell cycle checkpoint in various mammalian tissues ²¹⁻²⁴. In the MG, DNA damage occurs in alveolar cells during pregnancy ³⁵, however, whether it plays a physiological role during alveologenesis remains unknown. To determine the extent of DNA damage during alveologenesis and lactation, the phosphorylation of histone H2A.X at Serine-139 (γH2AX), a site that is rapidly phosphorylated in the presence of DNA strand breaks ³⁶, was investigated. γH2AX was found to be present in CK8+ luminal cells throughout alveologenesis, with the peak occurring at PD10.5 (FIG. 3a, b and Suppl. FIG. 4). In HC11 cells, γH2AX is highest in undifferentiated cells that are actively proliferating and lower in confluent cells during competency and priming (Suppl. FIG. 5a, b). However, an increase in γH2AX was detected when HC11 cells underwent differentiation (Suppl. FIG. 5a, b), and phalloidin staining shows that γH2AX accumulates in cells forming milk domes (Suppl. FIG. 5c). These results suggest DNA damage plays a role in lactogenic differentiation and endoreplication. To investigate, HC11 cells were treated with doxorubicin (50 nM), which induces DNA damage by inhibiting topoisomerase II during DNA replication. Accordingly, doxorubicin results in an increase of γH2AX 24 h post-treatment (Suppl. FIG. 6a, b). FACS DNA content analysis shows the proportion of tetraploid cells increases after 24 h of doxorubicin treatment, indicating the G2/M checkpoint was activated (Suppl. FIG. 6c, d). By DIP3, an increase in polyploidization is also detected (Suppl. FIG. 6e, f). Visualization of the FACS-purified population illustrates that DNA damage-induced endoreplication generated both mono- and binucleated polyploid cells, recapitulating the heterogeneity observed in the MG (Suppl. FIG. 6g). Polyploidization is accompanied by an increase of CSN2 detected by WB (Suppl. FIG. 6h).

To examine the in vivo consequences of damaging DNA during pregnancy, contralateral intraductal injection (IDI) of doxorubicin or DMSO-containing vehicle into MGs at PD12.5 was performed to extend the period of DNA damage that peaks at PD10 (FIG. 3a-c). Increased γH2AX was observed in CK8+ luminal cells 24 h post-injection with doxorubicin (FIG. 3d, e), demonstrating doxorubicin was effectively delivered into the MG epithelium. FACS DNA content analysis of doxorubicin-injected MGs at PD17.5 shows an increase in the proportion of 4C and >4C luminal populations (14.86% and 47.1% increase in the overall population, respectively), while the 2C population decreases (5.23% decrease in the overall population; FIG. 3f and Suppl. FIG. 6i, j). This increase in endoreplication is accompanied by an increase in milk production, as detected by RT-qPCR for the expression of Csn2 (FIG. 3g). Collectively, these results indicate DNA damage regulates alveolar endoreplication through the activation of the G2/M checkpoint and, consequently, milk production.

### Example 4—Replication Stress Results in Activation of the DNA Damage Response and Endoreplication During Alveologenesis

To ensure genomic stability and safeguard inheritance, cells possess a DNA damage response (DDR) that monitors genomic integrity throughout the cell cycle. During normal development, cell proliferation frequently results in activation of the DDR due to replication stress ^{37 38}. Given the tremendous amount of proliferation that occurs during early alveologenesis, it was hypothesized that replication stress may be the source of DNA damage driving endoreplication of mammary alveolar cells. The response to DNA damage by replication stress is mediated by the kinase ATR, which is activated by phosphorylation at Threonine-1989 (pATR) 39. By IHC staining, it was found, similarly to γH2AX (FIG. 3a, b and Suppl. FIG. 4), that ATR is activated at PD10.5 when proliferation is at its peak ⁴⁰ (FIG. 4a, b). However, while γH2AX decreases afterwards, pATR persists until the end of pregnancy, when polyploidization begins (FIG. 4a, b). To investigate the role of replication stress in alveolar endoreplication, it was induced in HC11 cells through over-expression of CYCLIN E (CCNE1) prior to lactogenic differentiation. CYCLIN E is an oncogene that accelerates DNA replication during the S phase of the cell cycle and results in DNA damage due to replication stress ^{41,42}. FACS analysis shows CCNE1 increases DNA replication, as measured by BrdU incorporation in HC11 cells. In accordance with DNA damage accumulation due to replication stress, γH2AX and pATR also increase (FIG. 4c, d). Additionally, FACS DNA content analysis shows CCNE1 increases endoreplication by DIP3, as well as CSN2 detected by WB (FIG. 4e, f). To investigate the effect of inducing replication stress in vivo, contralateral IDI of Hydroxyurea (Hu) or PBS vehicle into MGs at PD8.5 and PD12.5 was performed to encompass the peak of proliferation at PD10.5 ⁴⁰ (FIG. 5a), and detect increased γH2AX and pATR in the CK8+ population at PD13.5 by IHC staining (FIG. 5b-d). FACS analysis of DNA content in CK8+ cells reveals an increase in the 4C and >4C populations by LD5 (2% and 48% increase in the overall population, respectively), while the 2C population decreases (5.5% decrease in the overall population; FIG. 5e). This increase in endoreplication is accompanied by an increase in Csn2 expression as detected by RT-qPCR. Because areas of high and low milk staining in a ˜1 cm² section of LD2 MG tissue was observed, the entire section was imaged, milk staining contained within each alveolus was quantified, and the average integrated density among alveoli in the whole section was calculated. A ˜15% increase in milk in HU-injected MGs was observed (FIG. 5f-i). These results indicate that DNA damage produced by replication stress is sufficient to drive endoreplication during lactation.

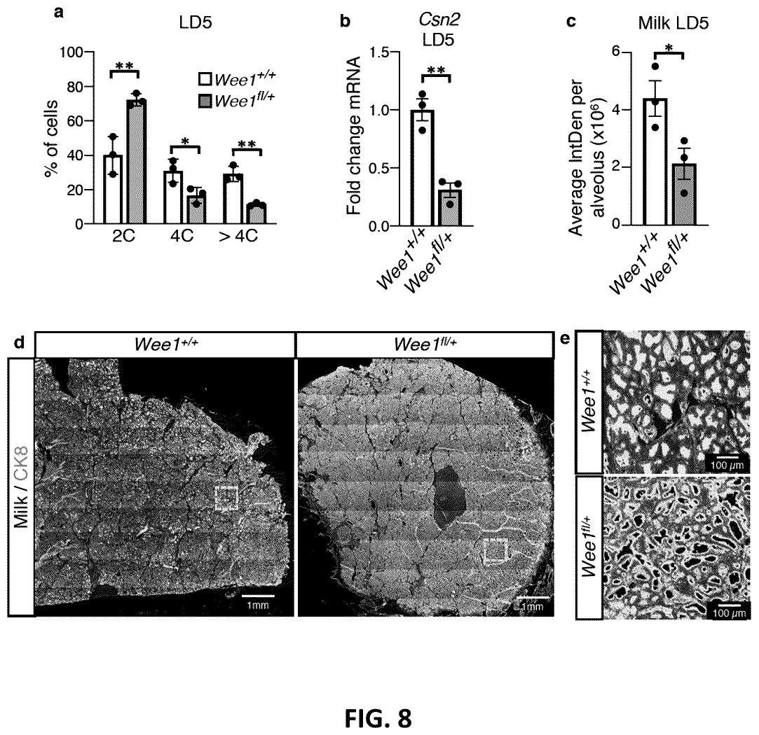

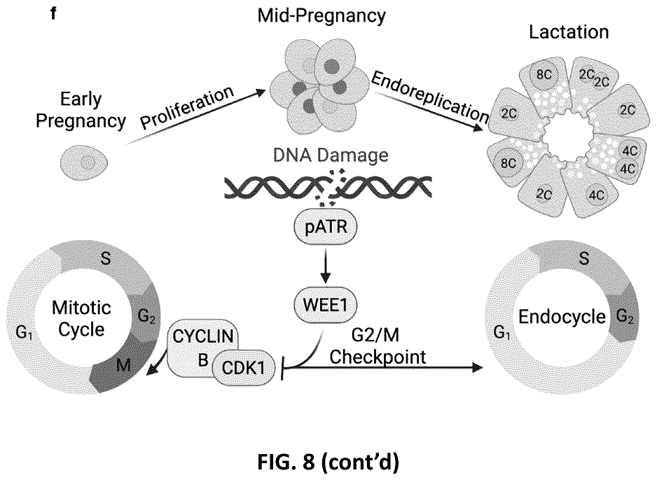

Previous studies have shown supplementation of nucleosides (Nucs) can relieve replication stress in cultured cells ^{42,43}. To investigate if nucleosides reduce replication stress in vivo, contralateral IDI of nucleosides or PBS vehicle into MGs at PD8.5 and PD12.5 were performed, again to encompass the peak of proliferation (FIG. 6a), and observe decreased γH2AX and pATR in the CK8+ population at PD13.5 by IHC staining (FIG. 5b-d). FACS analysis of DNA content in CK8+ luminal cells also reveals a decrease in the 4C population at LD2 (22.15% decrease in the overall population), and both the 4C and >4C populations by LD5 (6.05% and 14.79% decrease in the overall population, respectively; FIG. 6e, f and Suppl. FIG. 8c-f). Accordingly, the 2C population increases at both LD2 and LD5 (9% and 49% increase in the overall population, respectively; FIG. 6e, f and Suppl. FIG. 8c-f). These results suggest nucleoside IDI relieves replication stress during pregnancy and inhibits the activation of G2/M checkpoint at LD2, resulting in decreased endoreplication by LD5. This decrease in endoreplication is accompanied by a decrease in Csn2 expression and a 21% decrease in milk production, detected by RT-qPCR and IHC, respectively (FIG. 6g-j). Altogether, these findings strongly suggest replication stress during early alveologenesis causes DNA damage and leads to prolonged DDR. This, in turn, triggers the activation of the G2/M checkpoint and endoreplication of alveolar cells at the onset of lactation.

### Example 5—the DNA Damage Response Regulates Endoreplication Via WEE1

CDK1 inactivation during G2/M arrest can occur through several different inhibitors. The Cip and Kip family of CDK inhibitors, composed of P21^{Cip1}, P27^{Kip1} and P57^{Kip2}, is involved in the regulation of endoreplication ²⁸. In addition, the CDK1 inhibitor WEE1 is required for proper

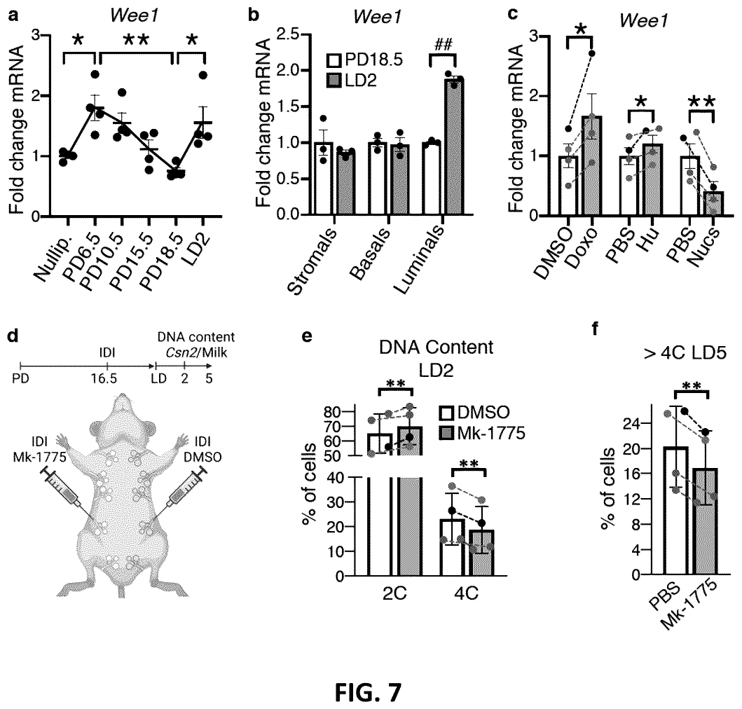

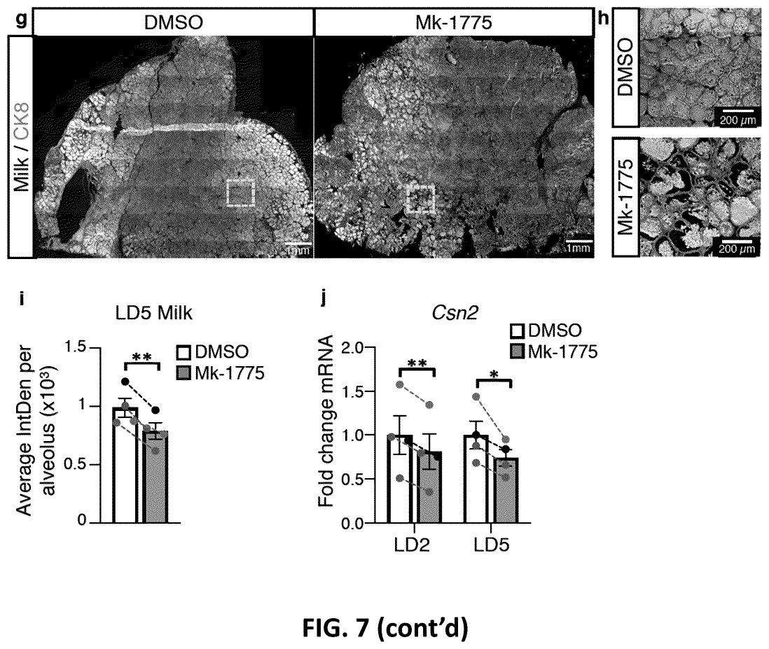

DNA replication and for the activation of the G2/M checkpoint in response to replication stress ^{44,45}. WEE1 also regulates endoreplication in plants ⁴⁶⁻⁴⁸. To determine which of these inhibitors may be regulating CDK1 activity during alveologenesis, their expression was analyzed during pregnancy and lactation by RT-qPCR. Expression of Cdkn1a and Cdkn1b, which encode for P21^{Cip1} and P27^{Kip1}, respectively, were found to remain unchanged in comparison to their expression in the nulliparous MG (Suppl. FIG. 9a). Conversely, Wee1 is upregulated during the cell proliferation occurring in early pregnancy, and again at LD2, during endoreplication (FIG. 7a). RT-qPCR of FACS-purified MG populations demonstrates Wee1 upregulation during lactation occurs specifically in the luminal population (FIG. 7b). Furthermore, Wee1 was found to be upregulated in response to IDI of doxorubicin and hydroxyurea into the pregnant MG and downregulated in response to IDI of nucleosides (FIG. 7c), demonstrating a direct correlation between Wee1 expression and the extent of endoreplication during alveologenesis. Consequently, the role of WEE1 was investigated during alveolar endoreplication in vivo by performing contralateral IDI of the WEE1 inhibitor Mk-1775 or DMSO-containing vehicle into MGs at PD16.5, to capture the beginning of endoreplication occurring by PD17.5 (FIG. 1f, 7d). FACS analysis of DNA content in CK8+ luminal cells reveals that injection of Mk-1775 decreases the 4C population at LD2 (19.4% decrease in the overall population), and both the 4C and >4C populations by LD5 (3.2% and 16.31% decrease in the overall population, respectively; FIG. 7e, f and Suppl. FIG. 9b-e). Accordingly, the 2C population increases at both LD2 and LD5 (7.7% and 12.4% increase in the overall population, respectively; FIG. 7e, f and Suppl. FIG. 9b-e). This decrease in endoreplication is accompanied by a decrease of Csn2 expression and a 21% decrease in milk production, detected by RT-qPCR and IHC, respectively (FIG. 7g-j).

Next, a Wee1 conditional knock-out mouse line was generated and the gene specifically deleted in luminal cells. Conditional deletion utilized a tamoxifen-inducible CreER system under the control of the Ck8 promoter, which also carries a mTmG reporter. Tamoxifen injections were performed at PD17.5 and LD2 to prevent potential deleterious effects caused by Wee1 loss during early alveologenesis. FACS analysis of GFP expression from Ck8-CreER/mTmG/Wee1^{fl/+} MGs at LD5 shows that recombination occurs specifically in the CK8⁺ population (Suppl. FIG. 10a, b). As expected, decreased Wee1 expression was detected by RT-qPCR (Suppl, FIG. 10c). FACS analysis of DNA content in CK8+ luminal cells at LD5 reveals a decrease in the 4C and >4C populations in the Ck8-CreER/mTmG/Wee1^{fl/+} MGs, while the 2C population increases (FIG. 8a and Suppl. FIG. 10d), showing that heterozygous loss of Wee1 is sufficient to prevent endoreplication. Accordingly, decreased Csn2 expression and a 52% decrease in milk production was detected by RT-qPCR and IHC, respectively (FIG. 8b-e). Together, these results demonstrate that WEE1 mediates the DDR response to replication stress by activating the G2/M checkpoint and regulating alveolar endoreplication and milk production (FIG. 8f).

### Methods

### Mouse Strains

CD-1 mice were obtained from Charles Rivers. C57BL/6-Wee1<tm1.1 mrl> were generated by Taconic. Ck8-CreER/mTmG mice were generously provided by Dr. Diwakar R Pattabiraman. These animals were a cross of Tg(Krt8-cre/ERT2)17Blpn/J (JAX:017947) and B6.129(Cg)-Gt(ROSA)26Sor^{tm4(ACTB-tdTomato, -EGFP)Luo}/J (JAX: 007676). Genotyping was performed by extracting DNA from ear snips and performing an end-point PCR for the given transgene using the primers CreER: 5′-CAGATGGCGCGGCAACACC-3′ (SEQ ID NO:1) and 5′-GCGCGGTCTGGCAGTAAAAAC-3′ (SEQ ID NO:2); mTmG: 5′-AGG GAG CTG CAG TGG AGT AG-3′ (SEQ ID NO:3), 5′-TAG AGC TTG CGG AAC CCT TC-3′ (SEQ ID NO:4) and 5′-CTT TAA GCC TGC CCA GAA GA-3′ (SEQ ID NO:5); Wee1: 5-GCTTCGGAACCTTCCTAATGC-3′ (SEQ ID NO:6) and 5′-TGAAGTCTCACCCTGTCTCG-3 (SEQ ID NO:7). All animal procedures were both approved by and conducted in accordance with the guidelines set by the University of California, Santa Cruz (UCSC) Institutional Animal Care and Use Committee (IACUC).

### Animal Studies

Nulliparous analysis was performed using adult (10-12-week-old) female mice. For timed pregnancies and lactation analysis, CD-1 pregnant adult females were obtained from Charles Rivers. Embryos were examined at the time of mammary gland harvesting to confirm pregnancy state. For lactation analysis on the C57BL/6-Wee1<tm1.1 mrl> mice, adult females were checked for the presence of a vaginal plug indicating that mating occurred. Plugged mice were considered to be PD0.5 on the day of the observed plug. All females used in this study were age matched.

### Contralateral Intraductal Injections

Mice were anesthetized with isoflurane (VetOne, 501017) Prior to intraductal injections, hair was removed from around the nipples using Nair hair remover. Injections into the duct of the nipple were performed with 33 G needles (Hamilton, 7803-05) and a volume of 40 μl per gland. Right inguinal, abdominal and thoracic glands were injected with doxorubicin (1.5 μg per gland, Cayman Chemical, 15007), nucleosides (40 μl per gland, Millipore-Sigma, ES-008-D) or Mk-1775 (50 μg per gland, Cayman Chemical, 21266), and left glands were injected with PBS or DMSO (Thermo Scientific, BP231) diluted in PBS.

### Tamoxifen Injections

Tamoxifen (Sigma, T5648) was dissolved in corn oil (Sigma, C8267) at a concentration of 20 mg/ml. Mice were anesthetized with isoflurane (VetOne, 501017). Intraperitoneal injections of 75 mg/kg bodyweight were performed at PD17.5 and LD2 using insulin syringes (Fisher Scientific, 14-826-79).

### Cell Cultures

The HC11 cell line was obtained from American Type Culture Collection (ATCC) and routinely checked for mycoplasma (Mycoplasma PCR kit, ABM, G238). Undifferentiated HC11 cells were cultured in growing medium (RPMI-1640; Thermo Fisher Scientific, 72400047), supplemented with 10% FBS, 5 μg/ml insulin (Millipore-Sigma, 16634), 10 ng/ml epidermal growth factor (EGF; Preprotech, AF-100-15) and 1× AntiAnti (Thermo Fisher Scientific, 15240112) at 37° C. with 5% CO2. Cells were grown to confluency and maintained in growing medium for 2 days, until they became competent. Competent HC11 cells were primed for differentiation by culturing them in priming medium [RPMI-1640 supplemented with 5 μg/ml insulin, 1 μM dexamethasone (Millipore-Sigma, D4902-1 G) and 1× Anti-Anti] for 24 h at 37° C. with 5% CO2. To induce differentiation, primed HC11 cells were cultured in DIP Medium [RPMI-1640, supplemented with 10% FBS, 5 μg/ml insulin, 1 μM dexamethasone, 1× anti-anti and 3 μg/ml Prolactin (NHPP, oPRL-21)] at 37° C. with 5% CO2.

For endoreplication studies, 80% confluent undifferentiated HC11 were treated with 30 μM blebbistatin (Millipore-Sigma, B0560), 5 μM Ro-3306 (Sigma Aldrich, SML0569), 100 nM doxorubicin (Cayman Chemical, 15007) or corresponding DMSO (Thermo Scientific, BP231) control. Drugs were maintained through the differentiation process and added with every change of media.

Undifferentiated HC11 were transfected with the Rc/CMV cyclin E plasmid (Addgene, #8963) for CCNE1 overexpression. Briefly, 70% confluent HC11 were transfected using Lipofectamine 3000 kit (Thermo Fisher, L3000015) and OPTI-MEM (GIBCO, 11058021). 48 h after transfection, HC11 were selected using 50 μg/ml of geneticin (Thermo Fisher, 10131035). Geneticin was maintained during HC11 culture and differentiation for stable CCNE1 expression.

### BrdU Incorporation Analysis

Undifferentiated HC11 were treated with 10 μM BrdU (Abcam, ab142567) for 2 h at 37° C. with 5% CO2. Cells were washed with 1×DPBS (GIBCO, 14190-250) and harvested using 0.05% Trypsin-EDTA (GIBCO, 25300-062). Cell suspension was washed with 1×DPBS and centrifuged at 1,000 rpm for 5 min at 4° C. Cell pellet was fixed in ice-cold 70% EtOH vortexing vigorously to avoid cell clumps. After fixation cells were washed with washing buffer [1×PBS (GIBCO, 14190136) containing 5% FBS] and centrifuged at 2,000 rpm for 5 min at 4° C. Cell pellet was treated with 500 μl of 2 M HCl for 20 min at room temperature. Cells were washed with 2 ml of 0.1 M sodium tetraborate (Sigma Aldrich, 221731) and centrifuged at 2,000 rpm for 5 min at 4° C. Cells were washed once more with 3 ml of 0.1M sodium tetraborate and one last time with 2 ml of washing buffer. Cells were incubated for 1 h at room temperature with anti-BrdU (Abcam, ab6326) or corresponding rat IgG (Thermo-Fisher, 10700) for isotype control. Cells were washed twice with 2 ml of washing buffer and incubated for 1 h at room temperature in darkness with FITC anti-Rat (Thermo Fisher, A24544). After incubation, cells were washed twice and resuspended in 500 μl of 1×PBS. Cells suspensions were filter through a 70 μm cell strainer (Falcon, 08-771-2) and analyzed using a BD LSRII cytometer. Populations were analyzed using FlowJo.

### Western Blotting

Whole cell lysates were prepared using 1×NP40 lysis buffer (Thermo Fisher Scientific, FNN0021) supplemented with Pierce Protease and Phosphatase inhibitors (Thermo Fisher Scientific, A32959). Cells were washed with ice-cold PBS (GIBCO, 14190136) and lysed direct in buffer and kept at 4° C. rocking at 70 rpm for 30 min. Lysed cells were collected and centrifuged at 12,000 rpm at 4° C. for 15 min. Protein concentration was quantified using Qubit 4 fluorometer (Thermo Fisher, Q33238). Samples were resolved by SDS page and transferred to polyvinylidene difluoride (PVDF, Millipore-Sigma, IPVH00010) for 60 min at 250 mA. Immunoblots were blocked for 1 h at room temperature using either 5% non-fat milk or 5% BSA TBST. Primary antibodies [anti-GAPDH (SCBT, sc-365062), anti-Actin (SCBT, sc-47778), anti-Cyclin B1 (SCBT, sc-245), anti-Cyclin E1 (Millipore-Sigma, SAB4503516) and anti-CSN2 (ABclonal, A12749)] were incubated overnight at 4° C. in a rocker. HRP-conjugated secondary antibodies (The Jackson Laboratory) were used for 1 h at room temperature. Immunoblots were developed using Clarity ECL (Bio-Rad), detected using a Bio-Rad ChemiDoc MP Image, and quantified using ImageJ.

### Mammary Gland Single Cell Suspension

Mechanically dissociated inguinal, abdominal, and thoracic mammary fat pads were prepared into cell suspension for flow cytometry or fluorescence-activated cell sorting (FACS). The lymph node was removed from abdominal glands. Glands were chopped using a mechanical tissue chopper and digested for 1 h at 37° C. in digestion media [RPM1 containing 1% FBS, collagenase IA (Sigma, C9891), hyaluronidase (Sigma, H3506) and DNAse I (Worthington, LS002007)]. Tissue was washed with washing buffer (1×PBS containing 2% FBS) and centrifuged at 1,000 rpm for 5 min at 4° C. Tissue was further digested using pre-warmed 0.25% Trypsin-EDTA (Thermo Fisher, 25200056), washed, and digested with 5 mg/ml of pre-warmed dispase II (Roche, 4942078001). Red blood cells were lysed using Ammonium Chloride Solution (Stem Cell Technologies, 07850). Cells were washed, resuspended and filter through a 70 μm cell strainer (Falcon, 08-771-2) and processed for downstream applications.

### Flow Cytometry

For DNA content analysis of the mammary gland CK8⁺ epithelial population, mammary gland cells suspension was obtained as described above. Cells were fixed in ice-cold 70% EtOH at a final concentration of 10⁶ cells/ml. During fixation, cells were vigorously vortexed for 1 min to avoid the formation of cell aggregates. Cells were washed twice with washing buffer [1×PBS containing 5% FBS and 0.5% tween 20 (Fisher Chemical, BP337500)] and centrifuged at 2,000 rpm for 5 min at 4° C. Cell pellet was incubated for 1 h at room temperature with anti-CK8 (Developmental Studies Hybridoma Lab, TROMA-1), anti-GFP (Thermo Fisher, A01704) or corresponding rat (Thermo-Fisher, 10700) or rabbit (Thermo-Fisher, 10500C) IgG isotype controls. Cells were washed twice and incubated for 1 h at room temperature in darkness with FITC anti-Rat (Thermo Fisher, A24544) or FITC anti-Rabbit (Thermo Fisher, A16030) and APC anti-Rat (Jackson ImmunoResearch, 712-136-153). Cells were washed twice and resuspended in propidium iodide solution [1×PBS containing 25 μg/ml of propidium iodide (Thermo Fisher, P3566) and 100 μg/ml of RNAse (Thermo Fisher, 12091021)]. For DNA content analysis on HC11, cells were washed with 1×DPBS (GIBCO, 14190-250) and harvested using 0.05% Trypsin-EDTA (GIBCO, 25300-062). Cell suspension was washed with 1×DPBS and centrifuged at 1,000 rpm for 5 min at 4° C. Cell pellet was fixed in ice-cold 70% EtOH and then vortexed vigorously to avoid cell aggregates. After fixation, cells were washed with washing buffer [1×PBS (GIBCO, 14190136) supplemented with 5% FBS] and centrifuged at 2,000 rpm for 5 min at 4° C. The pellet was resuspended in propidium iodide solution. Cell suspensions were filtered through a 70 μm cell strainer (Falcon, 08-771-2) and analyzed using a BD LSRII cytometer or a BD FACS Aria II Cell Sorter. Populations were analyzed using FlowJo.

### Visualization of Cells Sorted Based on DNA Content.

Mammary gland CK8+ epithelial cells or HC11 were sorted based on DNA content using BD FACS Aria II Cell Sorter. After sorting, cells were stained in suspension using Phalloidin-iFluor 488 Reagent (Abcam, ab176753) for 30 min at room temperature in darkness. Cells were spined down onto microscopy slides (Fisher, 12-550-15) using Cytospin 2 (Shandon, 599X52) at 500 rpm for 3 min. Cells were mounted using fluoromount-G (Southern Biotech, 0100-01) and visualized using Zeiss Axio Imager Microscope.

### Immunofluorescence on HC11 Cells

HC11 cells were fixed using ice-cold MeOH for 10 min. After washing with 1×PBS, cells were incubated for 1 h at room temperature with primary antibodies [anti-γH2AX (SCBT, sc-517348), anti-pATR (Genetex, GTX128145), anti-PLIN2 (generously provided by Jim McManaman)] in a humid incubation chamber. After incubation, cells were washed three times using 1×PBS and incubated for 1 h at room temperature in darkness using corresponding Alexa Fluor AffiniPure secondary antibodies (Jackson ImmunoResearch) and Phalloidin-iFluor 488 Reagent (Abcam, ab176753) when indicated. Cells were washed three times using 1×PBS and incubated with Hoechst 33342 (AnaSpec, AS-83218) for 10 min. Cells were mounted using fluoromount-G (Southern Biotech, 0100-01) and visualized using Zeiss Axio Imager Microscope. Integrated density of PLIN2, nuclear γH2AX and nuclear pATR was quantify using ImageJ.

### Immunofluorescence of Paraffin-Embedded Tissue

Mammary gland tissue was fixed in 10% neutral buffered formalin (EMD Millipore, MR0458682) at 4° C. overnight. Fixation was quenched using 0.2% glycine (Fisher Scientific, BP381) in PBS, for 1 h at room temperature. Tissue was dehydrated by incubating with 70% EtOH (Decon Labs, V1001) overnight, 95% EtOH for 1 h, 100% EtOH for 1 h (×3) and xylenes (Fisher Scientific, X3P) for 1 h (×3). Dehydrated tissue was soaked in paraffin (VWR, 15159-409) overnight and embedded. Paraffin-embedded tissue was sectioned at a thickness of 5 μm and mounted on Superfrost Plus Microscope Slides (Fisher, 12-550-15). Sectioned tissue was hydrated by incubating with xylenes for 5 min (×3), 100% ethanol for 2 min (×2), 95% ethanol for 1 min, 70% ethanol for 1 min, 50% ethanol for 1 min, and diH2O for 5 min. Antigen retrieval was performed using antigen unmasking solution (VectorLabs, H3300-250) in a conventional lab microwave. Sections were incubated with blocking buffer containing 10% donkey serum (Equitech-Bio, SD30), 1% BSA (VWR, 97061-422) and 0.3% triton (Millipore Sigma, X100) in PBS overnight at 4 C. Incubation with primary antibodies [anti-CK8 (Developmental Studies Hybridoma Lab, TROMA-1) and anti-mouse milk proteins (Accurate Chemical and Scientific, YNRM™)] was performed overnight at 4° C. Sections were washed with 0.3% triton in PBS for 30 min (×3) at room temperature. Incubation with secondary antibodies [(donkey anti-Rat 647 (Thermo-Invitrogen; A48272) and donkey anti-Rabbit 488 (Thermo-Invitrogen; A32790)] was performed for 2 h at room temperature. Sections were washed with 0.3% triton in PBS for 30 min (×3) at room temperature and mounted using flouromount-G (Southern Biotech, 0100-01). Image acquisition was performed using Zeiss Axio Imager Microscope. Macros were generated to quantify lumen milk integrated density per alveolus using Image J.

### Immunofluorescence and Optical Clearing of Cryosections

Mammary gland tissue was fixed in 10% neutral buffered formalin (EMD Millipore, MR0458682) at 4° C. overnight. Fixation was quenched using 0.2% glycine (Fisher Scientific, BP381) in PBS, for 1 h at room temperature. Tissue was incubated with 30% sucrose (Fisher Scientific, BP220-212) in PBS at 4° C. for 48 h and sectioned at a thickness of 100 μm. Sections were washed with PBS for 10 min (×2) at room temperature. Sections were incubated with CUBIC-L⁵⁴ at 37 C overnight, and washed with 0.3% triton in PBS for 30 min (×3). Sections were incubated with blocking buffer containing 10% donkey serum (Equitech-Bio, SD30), 1% BSA (VWR, 97061-422) and 0.3% triton (Millipore Sigma, X100) in PBS overnight at 4° C. Incubation with primary antibodies [anti-γH2AX (Cell Signaling, 2577S), anti-pATR (Genetex, GTX128145), anti-CK8 (Developmental Studies Hybridoma Lab, TROMA-1)] was performed overnight at 4° C. Sections were washed with 0.3% triton in PBS for 1 h (×3) at room temperature. Incubation with secondary antibodies [donkey anti-Rat 488 (Thermo-Invitrogen; A32795) and donkey anti-Rabbit 647 (Thermo-Invitrogen; A48269)], propidium iodide (Thermo Fisher, P3566) and RNAse (Thermo Fisher, 12091021) was performed for 6 h at room temperature. Sections were washed with 0.3% triton in PBS for 1 h (×3) at room temperature and mounted on poly-L-lysine (Sigma, P8920) coated chamber slides (Ibidi, 80827). Tissues sections were incubated with CUBIC-R⁵⁴ at room temperature until cleared (approximately 48 h), and imaged using a ZEISS LSM 880 microscope with Airyscan. Integrated density of nuclear γH2AX and pATR was quantified using ImageJ.

### 3D DNA Content In Situ

Mammary gland tissue was fixed in 10% neutral buffered formalin (EMD Millipore, MR0458682) at 4° C. overnight. Fixation was quenched using 0.2% glycine (Fisher Scientific, BP381) in PBS, for 1 h at room temperature. Tissue was incubated with 30% sucrose (Fisher Scientific, BP220-212) in PBS at 4° C. for 48 h and sectioned at a thickness of 200 μm. Sections were washed with PBS for 10 min (×2) at room temperature. Sections were incubated with blocking buffer containing 10% donkey serum (Equitech-Bio, SD30), 1% BSA (VWR, 97061-422) and 0.3% triton (Millipore Sigma, X100) in PBS overnight at 4° C. Incubation with primary antibody anti-E-cadherin (Thermo Fisher, 13-1900) was performed overnight at 4° C. Sections were washed with 0.3% triton in PBS for 1 h (×3) at room temperature. Incubation with secondary antibody donkey anti-Rat 488 (Thermo-Invitrogen; A32795), Phalloidin-647 (Invitrogen, A30107) and Hoechst 33342 (AnaSpec, AS-83218) was performed overnight at 4° C. Sections were washed with 0.3% triton in PBS for 1 h (×3) at room temperature and mounted on poly-L-lysine (Sigma, P8920) coated chamber slides (Ibidi, 80827). Tissue sections were incubated 80% glycerol (Sigma, G9012) in H2O at room temperature for 72 h, and imaged using a ZEISS LSM 880 microscope with Airyscan. Segmentation of nuclei and quantification of DNA integrated density in 3D was performed using Cell Profiler. Incomplete nuclei were excluded from analysis. Stromal cells were used as a reference for diploid (2C) DNA content.

### RNA Extraction and RT-qPCR

For RNA isolation from FACS purified populations, mammary gland cell suspensions were blocked using Mouse BD Fc Block™ (BD Biosciences) for 10 min. Cells were subsequently resuspended on 1×PBS at a density of 10⁷ cells/ml and stained with the following antibodies for 30 min on ice: anti-CD24 PE (Stem Cell Technologies, 60099PE.1), anti-CD29 PE-Cy7 (BioLegend, 102222), anti-CD45-APC (BioLegend,105826), Ter119-APC (BD Biosciences, 561033), CD31-ACP (BD Biosciences, 551262). Propidium iodide at a final concentration of 0.5 μg/ml was used for the discrimination of dead cells. Stromal, basal and luminal mammary populations were sorted using a BD FACS Aria II Cell Sorter. Cells were subsequently lysed in TRIzol reagent (ThermoFisher, 15596018) and phase separated according to the manufacturer's protocol with an additional overnight RNA precipitation step in ethanol ⁵⁵. The RNA was further purified with TURBO DNase (Ambion, AM1906) treatment. For HC11 and whole-gland tissue RNA isolation the NucleoSpin RNA extraction kit (Macherey-Nagel, 740955.50) was utilized according to the manufacturer's instructions. Total RNA quality was analyzed by agarose gel electrophoresis and quantified using an ND-1000 spectrophotometer (NanoDrop). cDNA was prepared from 500-1000 ng of total RNA using iScript cDNA synthesis kit (Bio-Rad, 1708841). Quantitative RT-qPCR was performed in triplicates using SsoAdvanced Universal SYBR Green Supermix, (Bio-Rad, 1725272). The reactions were run in a Bio-Rad CFX'Connect Real-Time System and CFX Manager software (Bio-Rad) as follows: 95° C. for 2 min followed by 40 cycles of 95° C. for 15 s, 60° C. for 30 s and 72° C. for 45 s. Results were normalized to Gapdh. Primers used in this study are: Csn2: 5′-CCTCTGAGACTGATAGTATT-3′ (SEQ ID NO:8) and 5′-TGGATGCTGGAGTGAACTTTA-3′ (SEQ ID NO:9); Gapdh: 5′-CATGGCCTTCCGTGTTCCTA-3′ (SEQ ID NO:10) and 5′-CCTGCTTCACCACCTTCTTGAT-3′ (SEQ ID NO:11); Cdkn1a: 5′-ATCCAGACATTCAGAGCCACAG-3′ (SEQ ID NO:12) and 5′-ACGAAGTCAAAGTTCCACCGT-3′ (SEQ ID NO:13); Wee1: 5′-TTGGCTGGCTCTGTTGATGA-3′ (SEQ ID NO:14) and 5′-CAGCTAAACTCCCACCATTACAG-3′ (SEQ ID NO:15); Cdkn1b: 5′-AACGTGCGAGTGTCTAACGG-3′ (SEQ ID NO:16) and 5′-CCCTCTAGGGGTTTGTGATTCT-3′ (SEQ ID NO:17).

### Statistical Analyses

No statistical method was used to predetermine sample size. Statistical analysis was performed using Prism9 software. Sample size, biological replicates, statistical test, and statistical significance are denoted in the figure legends. For the statistical analysis of the experiments involving contralateral intraductal injections, paired statistical tests were performed.

### REFERENCES

1 Gardner, R. L. & Davies, T. J. Lack of coupling between onset of giant transformation and genome endoreduplication in the mural trophectoderm of the mouse blastocyst. J Exp Zool 265, 54-60 (1993). https://doi.org:10.1002/jez.1402650108
2 MacAuley, A., Cross, J. C. & Werb, Z. Reprogramming the cell cycle for endoreduplication in rodent trophoblast cells. Mol Biol Cel/9, 795-807 (1998). https://doi.org:10.1091/mbc.9.4.795
3 Hemberger, M., Hanna, C. W. & Dean, W. Mechanisms of early placental development in mouse and humans. Nat Rev Genet 21, 27-43 (2020). https://doi.org:10.1038/s41576-019-0169-4
4 Kirk, D. & Clingan, D. A. Normal human endometrium in cell culture. Ill. Mechanism(s) of epithelial polyploidization. Cell Biol Int Rep 4, 83-92 (1980). https://doi.org:10.1016/0309-1651(80)90013-2
Ansell, J. D., Barlow, P. W. & McLaren, A. Binucleate and polyploid cells in the decidua of the mouse. J Embryo Exp Morphol 31, 223-227 (1974).
6 SACHS, L. & SHELESNYAK, M. C. The development and suppression of polyploidy in the developing and suppressed deciduoma in the rat. J Endocrinol 12, 146-151 (1955). https://doi.org:10.1677/joe.0.0120146
7 Milona, A. et al. The normal mechanisms of pregnancy-induced liver growth are not maintained in mice lacking the bile acid sensor Fxr. Am J Physiol Gastrointest Liver Physiol 298, G151-158 (2010). https://doi.org:10.1152/ajpgi.00336.2009
8 Banerjee, M. R., Wagner, J. E. & Kinder, D. L. DNA synthesis in the absence of cell reproduction during functional differentiation of mouse mammary gland. Life Sci II 10, 867-877 (1971).
9 Banerjee, M. R. & Wagner, J. E. Gene amplification in mammary gland at differentiation. Biochem Biophys Res Commun 49, 480-487 (1972). https://doi.org:10.1016/0006-291x(72)90436-6
10 Smith, G. H. & Vonderhaar, B. K. Functional differentiation in mouse mammary gland epithelium is attained through DNA synthesis, inconsequent of mitosis. Dev Biol 88, 167-179 (1981).
11 Rios, A. C. et al. Essential role for a novel population of binucleated mammary epithelial cells in lactation. Nat Commun 7, 11400 (2016). https://doi.org:10.1038/ncomms11400
12 Macias, H. & Hinck, L. Mammary gland development. Wiley Interdiscip Rev Dev Biol 1, 533-557 (2012). https://doi.org:10.1002/wdev.35
13 Ullah, Z., Lee, C. Y., Lilly, M. A. & DePamphilis, M. L. Developmentally programmed endoreduplication in animals. Cell Cycle 8, 1501-1509 (2009). https://doi.org:10.4161/cc.8.10.8325
14 Zhang, Y., Wang, Z. & Ravid, K. The Cell Cycle in Polyploid Megakaryocytes Is Associated with Reduced Activity of Cyclin B1-dependent Cdc2 Kinase. Journal of Biological Chemistry 271, 4266-4272 (1996). https://doi.org:10.1074/jbc.271.8.4266
15 Ullah, Z., Kohn, M. J., Yagi, R., Vassilev, L. T. & Depamphilis, M. L. Differentiation of trophoblast stem cells into giant cells is triggered by p57/Kip2 inhibition of CDK1 activity. Genes & Development 22, 3024-3036 (2008). https://doi.org:10.1101/gad.1718108
16 Tan, J. et al. Evidence for coordinated interaction of cyclin D3 with p21 and cdk6 in directing the development of uterine stromal cell decidualization and polyploidy during implantation. Mech Dev 111, 99-113 (2002). https://doi.org:10.1016/s0925-4773(01)00614-1
17 Margall-Ducos, G., Celton-Morizur, S. V., Couton, D., BréGerie, O. & Desdouets, C. Liver tetraploidization is controlled by a new process of incomplete cytokinesis. Journal of Cell Science 120, 3633-3639 (2007). https://doi.org:10.1242/jcs.016907
18 Leone, M., Musa, G. & Engel, F. B. Cardiomyocyte binucleation is associated with aberrant mitotic microtubule distribution, mislocalization of RhoA and IQGAP3, as well as defective actomyosin ring anchorage and cleavage furrow ingression. Cardiovascular Research 114, 1115-1131 (2018). https://doi.org:10.1093/cvr/cvy056
19 Reboutier, D., Benaud, C. & Prigent, C. Aurora A's Functions During Mitotic Exit: The Guess Who Game. Front Oncol5, 290 (2015). https://doi.org:10.3389/fonc.2015.00290
20 Sancar, A., Lindsey-Boltz, L. A., Unsal-Kaçmaz, K. & Linn, S. Molecular mechanisms of mammalian DNA repair and the DNA damage checkpoints. Annu Rev Biochem 73, 39-85 (2004). https://doi.org:10.1146/annurev.biochem.73.011303.073723
21 de Pedro, I., Alonso-Lecue, P., Sanz-Gómez, N., Freije, A. & Gandarillas, A. Sublethal UV irradiation induces squamous differentiation via a p53-independent, DNA damage-mitosis checkpoint. Cell Death Dis 9, 1094 (2018). https://doi.org:10.1038/s41419-018-1130-8
22 Sanz-Gómez, N. et al. Response of head and neck epithelial cells to a DNA damage-differentiation checkpoint involving polyploidization. Head Neck 40, 2487-2497 (2018). https://doi.org:10.1002/hed.25376
23 Freije, A. et al. Inactivation of p53 in Human Keratinocytes Leads to Squamous Differentiation and Shedding via Replication Stress and Mitotic Slippage. Cell reports 9, 1349-1360 (2014). https://doi.org:10.1016/j.celrep.2014.10.012
24 Molinuevo, R. et al. FOXM1 allows human keratinocytes to bypass the oncogene-induced differentiation checkpoint in response to gain of MYC or loss of p53. Oncogene 36, 956-965 (2017). https://doi.org:10.1038/onc.2016.262
25 Ho, T. L. F., Guilbaud, G., Blow, J. J., Sale, J. E. & Watson, C. J. The KRAB Zinc Finger Protein Roma/Zfp157 Is a Critical Regulator of Cell-Cycle Progression and Genomic Stability. Cell Rep 15, 724-734 (2016). https://doi.org:10.1016/j.celrep.2016.03.078
26 Sornapudi, T. R. et al. Comprehensive profiling of transcriptional networks specific for lactogenic differentiation of HC11 mammary epithelial stem-like cells. Sci Rep 8, 11777 (2018). https://doi.org:10.1038/s41598-018-30122-4
27 Straight, A. F. et al. Dissecting temporal and spatial control of cytokinesis with a myosin II Inhibitor. Science 299, 1743-1747 (2003). https://doi.org:10.1126/science.1081412
28 Ullah, Z., Lee, C. Y. & Depamphilis, M. L. Cip/Kip cyclin-dependent protein kinase inhibitors and the road to polyploidy. Cell Div4, 10 (2009). https://doi.org:10.1186/1747-1028-4-10
29 Øvrebo, J. I. & Edgar, B. A. Polyploidy in tissue homeostasis and regeneration. Development 145 (2018). https://doi.org:10.1242/dev.156034
30 Senyo, S. E. et al. Mammalian heart renewal by pre-existing cardiomyocytes. Nature 493, 433-436 (2013). https://doi.org:10.1038/nature 11682
31 Diril, M. K. et al. Cyclin-dependent kinase 1 (Cdk1) is essential for cell division and suppression of DNA re-replication but not for liver regeneration. Proc Natl Acad Sci USA 109, 3826-3831 (2012). https://doi.org:10.1073/pnas.1115201109
32 Miyaoka, Y. et al. Hypertrophy and unconventional cell division of hepatocytes underlie liver regeneration. Curr Biol 22, 1166-1175 (2012). https://doi.org:10.1016/j.cub.2012.05.016
33 Lazzeri, E. et al. Endocycle-related tubular cell hypertrophy and progenitor proliferation recover renal function after acute kidney injury. Nat Commun 9, 1344 (2018). https://doi.org:10.1038/s41467-018-03753-4
34 Herrtwich, L. et al. DNA Damage Signaling Instructs Polyploid Macrophage Fate in Granulomas. Cell 167, 1264-1280.e1218 (2016). https://doi.org:10.1016/j.cell.2016.09.054
35 Xu, X. et al. BRCA1 represses DNA replication initiation through antagonizing estrogen signaling and maintains genome stability in parallel with WEE1-MCM2 signaling during pregnancy. Hum Mol Genet 28, 842-857 (2019). https://doi.org:10.1093/hmg/ddy398
36 Fernandez-Capetillo, O., Lee, A., Nussenzweig, M. & Nussenzweig, A. H2AX: the histone guardian of the genome. DNA Repair (Amst) 3, 959-967 (2004). https://doi.org:10.1016/j.dnarep.2004.03.024
37 Miermont, A. et al. The fate of cells undergoing spontaneous DNA damage during development. Development 146 (2019). https://doi.org:10.1242/dev.174268
38 Zeman, M. K. & Cimprich, K. A. Causes and consequences of replication stress. Nat Cell Bio/16, 2-9 (2014). https://doi.org:10.1038/ncb2897
39 Nam, E. A. et al. Thr-1989 phosphorylation is a marker of active ataxia telangiectasia-mutated and Rad3-related (ATR) kinase. J Biol Chem 286, 28707-28714 (2011). https://doi.org:10.1074/jbc.M 111.248914
40 Richert, M. M., Schwertfeger, K. L., Ryder, J. W. & Anderson, S. M. An atlas of mouse mammary gland development. J Mammary Gland Biol Neoplasia 5, 227-241 (2000). https://doi.org:10.1023/a:1026499523505
41 Jones, R. M. et al. Increased replication initiation and conflicts with transcription underlie Cyclin E-induced replication stress. Oncogene 32, 3744-3753 (2013). https://doi.org:10.1038/onc.2012.387
42 Bester, A. C. et al. Nucleotide deficiency promotes genomic instability in early stages of cancer development. Cell 145, 435-446 (2011). https://doi.org:10.1016/j.cell.2011.03.044
43 Halliwell, J. A. et al. Nucleosides Rescue Replication-Mediated Genome Instability of Human Pluripotent Stem Cells. Stem Cell Reports 14, 1009-1017 (2020). https://doi.org:10.1016/j.stemcr.2020.04.004
44 Smith, H. L., Southgate, H., Tweddle, D. A. & Curtin, N. J. DNA damage checkpoint kinases in cancer. Expert Rev Mol Med 22, e2 (2020). https://doi.org:10.1017/erm.2020.3
45 Elbok, C. R., Petrosius, V. & Sorensen, C. S. WEE1 kinase limits CDK activities to safeguard DNA replication and mitotic entry. Mutat Res 819-820, 111694 (2020). https://doi.org:10.1016/j.mrfmmm.2020.111694
46 Chevalier, C. et al. Elucidating the functional role of endoreduplication in tomato fruit development. Ann Bot 107, 1159-1169 (2011). https://doi.org:10.1093/aob/mcq257
47 Sun, Y. et al. Characterization of maize (Zea mays L.) Wee1 and its activity in developing endosperm. Proc Natl Acad Sci USA 96, 4180-4185 (1999). https://doi.org:10.1073/pnas.96.7.4180
48 Gonzalez, N., Gevaudant, F., Hernould, M., Chevalier, C. & Mouras, A. The cell cycle-associated protein kinase WEE1 regulates cell size in relation to endoreduplication in developing tomato fruit. Plant J. 51, 642-655 (2007). https://doi.org:10.1111/j.1365-313X.2007.03167.x
49 Zanet, J. et al. A mitosis block links active cell cycle with human epidermal differentiation and results in endoreplication. PloS one 5, e15701 (2010). https://doi.org:10.1371/journal.pone.0015701
50 González-Rosa, J. M. et al. Myocardial Polyploidization Creates a Barrier to Heart Regeneration in Zebrafish. Dev Cell 44, 433-446.e437 (2018). https://doi.org:10.1016/j.devcel.2018.01.021
51 Chowdhury, R. et al. Breastfeeding and maternal health outcomes: a systematic review and meta-analysis. Acta Paediatr. 104, 96-113 (2015). https://doi.org:10.1111/apa.13102
52 Victora, C. G. et al. Breastfeeding in the 21 st century: epidemiology, mechanisms, and lifelong effect. Lancet 387, 475-490 (2016). https://doi.org:10.1016/S0140-6736(15)01024-7
53 Sewell, C. A., Chang, C. Y., Chehab, M. M. & Nguyen, C. P. Domperidone for Lactation: What Health Care Providers Need to Know. Obstet. Gynecol. 129, 1054-1058 (2017). https://doi.org:10.1097/aog.0000000000002033
54 Tainaka, K. et al. Chemical Landscape for Tissue Clearing Based on Hydrophilic Reagents. Cell Rep 24, 2196-2210.e2199 (2018). https://doi.org:10.1016/j.celrep.2018.07.056
55 Macias, H. et al. SLIT/ROBO1 signaling suppresses mammary branching morphogenesis by limiting basal cell number. Dev Cell 20, 827-840 (2011). https://doi.org:10.1016/j.devcel.2011.05.012

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method for enhancing milk production in a lactating mammalian subject, the method comprising:
administering a CDK1 inhibitor to mammary gland alveolar cells of the subject in an amount effective to enhance milk production in the lactating mammalian subject.

2. The method according to claim 1, wherein the CDK1 inhibitor is an agent that increases expression of Wee1-like protein kinase (WEE1) as compared to WEE1 expression in the absence of the agent.

3. The method according to claim 1, wherein the CDK1 inhibitor is a DNA damaging agent.

4. The method according to claim 3, wherein the DNA damaging agent is a DNA intercalating agent.

5. The method according to claim 4, wherein the DNA intercalating agent is an anthracycline.

6. The method according to claim 5, wherein the anthracycline is doxorubicin.

7. The method according to claim 3, wherein the DNA damaging agent is an inhibitor of ribonucleoside diphosphate reductase.

8. The method according to claim 7, wherein the DNA damaging agent is hydroxyurea.

9. The method according to claim 1, wherein the CDK1 inhibitor is an agent that reduces expression of CDK1.

10. The method according to claim 9, wherein the CDK1 inhibitor is a nucleic acid-based inhibitor comprising a region complementary to a portion of a messenger RNA (mRNA) that encodes CDK1.

11. The method according to claim 10, wherein the nucleic acid-based inhibitor is a short interfering RNA (siRNA), a microRNA (miRNA), or a morpholino.

12. The method according to claim 1, wherein the CDK1 inhibitor is parenterally administered to the lactating mammalian subject.

13. The method according to claim 12, wherein the CDK1 inhibitor is intraductally administered to the lactating mammalian subject.

14. The method according to claim 1, wherein the lactating mammalian subject is a lactating human subject.

15. The method according to claim 1, wherein the lactating mammalian subject is a lactating bovine subject.

16. The method according to claim 1, wherein the lactating mammalian subject is a lactating ovine, caprine, or camelid subject.


SEQUENCE LISTING (17 sequences)

SEQ ID NO: 1 - DNA, 19 bp, synthetic construct
  source 1..19: mol_type = other DNA
cagatggcgcggcaacacc

SEQ ID NO: 2 - DNA, 21 bp, synthetic construct
  source 1..21: mol_type = other DNA
gcgcggtctggcagtaaaaac

SEQ ID NO: 3 - DNA, 20 bp, synthetic construct
  source 1..20: mol_type = other DNA
agggagctgcagtggagtag

SEQ ID NO: 4 - DNA, 20 bp, synthetic construct
  source 1..20: mol_type = other DNA
tagagcttgcggaacccttc

SEQ ID NO: 5 - DNA, 18 bp, synthetic construct
  source 1..18: mol_type = other DNA
ctttaagcctgcccagaa

SEQ ID NO: 6 - DNA, 21 bp, synthetic construct
  source 1..21: mol_type = other DNA
gcttcggaaccttcctaatgc

SEQ ID NO: 7 - DNA, 20 bp, synthetic construct
  source 1..20: mol_type = other DNA
tgaagtctcaccctgtctcg

SEQ ID NO: 8 - DNA, 20 bp, synthetic construct
  source 1..20: mol_type = other DNA
cctctgagactgatagtatt

SEQ ID NO: 9 - DNA, 21 bp, synthetic construct
  source 1..21: mol_type = other DNA
tggatgctggagtgaacttta

SEQ ID NO: 10 - DNA, 20 bp, synthetic construct
  source 1..20: mol_type = other DNA
catggccttccgtgttccta

SEQ ID NO: 11 - DNA, 22 bp, synthetic construct
  source 1..22: mol_type = other DNA
cctgcttcaccaccttcttgat

SEQ ID NO: 12 - DNA, 22 bp, synthetic construct
  source 1..22: mol_type = other DNA
atccagacattcagagccacag

SEQ ID NO: 13 - DNA, 21 bp, synthetic construct
  source 1..21: mol_type = other DNA
acgaagtcaaagttccaccgt

SEQ ID NO: 14 - DNA, 20 bp, synthetic construct
  source 1..20: mol_type = other DNA
ttggctggctctgttgatga

SEQ ID NO: 15 - DNA, 23 bp, synthetic construct
  source 1..23: mol_type = other DNA
cagctaaactcccaccattacag

SEQ ID NO: 16 - DNA, 20 bp, synthetic construct
  source 1..20: mol_type = other DNA
aacgtgcgagtgtctaacgg

SEQ ID NO: 17 - DNA, 22 bp, synthetic construct
  source 1..22: mol_type = other DNA
ccctctaggggtttgtgattct